SEQUENCE LISTING (10 sequences)

SEQ ID NO: 1 - protein, 328 aa, Homo sapiens
  REGION 1..328: note = misc_feature - IL-12 p40 (Uniprot P29460)
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 307 aa, synthetic construct
  REGION 1..307: note = Synthetic: IL-12 p40 (mature form with N-terminal methionine)
MIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 198 aa, synthetic construct
  REGION 1..198: note = Synthetic: IL-12 p35 (mature form with N-terminal methionine)
MRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 253 aa, Homo sapiens
  REGION 1..253: note = misc_feature - IL-12 p35 (NP_000873.2)
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 239 aa, Homo sapiens
  REGION 1..239: note = misc_feature - IL-12 p35 (NP_001341511.1)
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 215 aa, Homo sapiens
  REGION 1..215: note = misc_feature - IL-12 p35 (NP_001341512.1)
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - protein, 317 aa, synthetic construct
  REGION 1..317: note = Synthetic: IL-12 p40 (mature form with N-terminal methionine and C-terminal 6His)
MIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSHHHHHH

SEQ ID NO: 8 - protein, 317 aa, synthetic construct
  REGION 1..317: note = Synthetic: IL-12P40-HIS6_C253S
MIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFSVQVQGKSKREKKDRVFTDKTSATVICRKNASISVRAQDRYYSSSWSEWASVPCSGGGSHHHHHH

SEQ ID NO: 9 - protein, 198 aa, synthetic construct
  REGION 1..198: note = Synthetic: IL-12P35_C16S_C89S
MRNLPVATPDPGMFPSLHHSQNLLRAVSNMLQKARQTLEFYPCTSEEIDHEDITKDKTSTVEACLPLELTKNESCLNSRETSFITNGSSLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 4 aa, synthetic construct
  REGION 1..4: note = Synthetic: linker
GGGS

(19) United States
(12) Patent Application Publication
(10) Pub. No.: US 2026/0295010 A1
(43) Pub. Date: Oct. 1, 2026

(54) IL-12 MUTANTS WITH REDUCED TOXICITY, COMPOSITIONS THEREOF AND METHODS OF USING THE SAME

(71) Applicant: Sutro Biopharma, Inc., South San Francisco, CA (US)

(72) Inventors: Junhao YANG, Palo Alto, CA (US); Amandeep Kaur GAKHAL, San Mateo, CA (US); Alice YAM, Belmont, CA (US); Daniel CALARESE, Millbrae, CA (US); Robert HENNINGSEN, Redwood City, CA (US)

(21) Appl. No.: 18/880,512

(22) PCT Filed: Jun. 30, 2023
(86) PCT No.: PCT/US2023/026802

§ 371 Date: Dec. 31, 2024

Related U.S. Application Data
(60) Provisional application No. 63/357,939, filed on Jul. 1, 2022.

(51) Int. Cl. A61K 38/20; A61K 38/00; A61K 39/395; C07K 14/54; C07K 14/55

(52) U.S. Cl. CPC A61K 38/208; A61K 38/2013; A61K 39/3955; C07K 14/5434; C07K 14/55; A61K 38/00

(57) ABSTRACT

The present disclosure provided to IL-12 polypeptides with at least one amino acid substitution, pharmaceutical compositions, diagnostic compositions, and kits containing the polypeptides, nucleic acids and expression vectors encoding the polypeptides, cells comprising the same, and methods of using the polypeptides, nucleic acids, expression vectors, and cells for therapeutic and diagnostic purposes.

### CROSS-REFERENCE TO RELATED APPLICATIONS

This application claims the benefit of, and priority to, U.S. Provisional Patent Application No. 63/357,939, filed Jul. 1, 2022, which is incorporated by reference herein in its entirety.

### FIELD OF THE INVENTION

The present disclosure generally relates to IL-12 polypeptides with one or more mutations, which illustrate improved effects (e.g., reduced receptor binding and/or reduced toxicity). Also provided are pharmaceutical compositions, diagnostic compositions and kits containing the polypeptides disclosed herein, nucleic acids and expression vectors encoding the polypeptides disclosed herein, cells comprising the same, and methods of using the polypeptides, nucleic acids, expression vectors, and cells for therapeutic, and diagnostic purposes.

### BACKGROUND

During the past two decades, interleukin-12 (IL-12) has emerged as one of the most potent cytokines in mediating antitumor activity in a variety of preclinical models. However, short half-life in circulation and toxicity have limited its application in clinical trial of cancer treatment (Tugues et al., 2015, Cell Death and Differentiation 22:237-246).

IL-12 is a heterodimeric cytokine having an α-subunit (p35; IL-12p35) and a β-subunit (p40; IL-12p40) (Wolf et al., 1994, Stem Cells 12:154-168). IL-12 binds a heterodimeric IL-12 receptor complex formed by IL-12 receptor β1 (IL-12RB1) and IL-12 receptor β2 (IL-12RB2) (Pressky et al., Proc. Natl. Acad. Sci. USA 93:14002-14007). IL-12 is reportedly involved in the differentiation of naïve T cells into TH1 cells, to stimulate the growth and function of T cells, to stimulate the production of IFNγ and TNFα from T cells and natural killer cells, and to reduce IL-4 mediated suppression of IFNγ (Hsieh et al., 1993, Science 260:547-549; Zheng et al., 2016, Regulation of Cytokine Gene Expression in Immunity and Diseases 941:117-138). An IL-12 p40 homodimer has also been characterized (Mondal et al., 2020, Proc. Natl. Acad. Sci. USA 117:21557-21567). IL-12 has been proposed for therapy with limited success (Nguyen et al., 2020, Front. Immunol. 11:1-36).

To overcome short half-life and toxicity, IL-12-Fc fusion scaffolds have been proposed to extend cytokine half-life, as well as novel IL-12 variants with decreased potency (US 2020/0216509 A1). Further, certain IL-12 p40 Ala variants that mediate interactions with Rb1 were shown to attenuate toxicity (Glassman et al., 2021, Cell 184:983-999). Accordingly, IL-12 variants with reduced toxicity would enable further development of IL-12 as a therapeutic.

### SUMMARY

Provided herein are variant IL-12 polypeptides comprising one or more mutations described herein. In certain embodiments, the variant IL-12 polypeptides provide reduced receptor binding. In certain embodiments, the variant IL-12 polypeptides provide reduced toxicity. In certain embodiments, the variant IL-12 polypeptides provide reduced receptor binding and reduced toxicity.

In one aspect, provided herein are variant IL-12 p40 subunit polypeptides. In certain embodiments, the variant IL-12 p40 subunit polypeptides comprise at least one amino acid substitution selected from the group consisting of: W16X, Y17A, P21A, D42A, D42K, Q43A, S44A, S45A, E46A, E46K, K59A, K59E, K85E, E87A, E87K, D88A, D88K, 190A, W91A, D94X, K103A, K103E, N104A, K105A, K105E, D162A, D162K, H195A, H195E, K196A, K196E, L197A, K198E, N201A, T203A, C253X, K259Y, K265Y, and I278Y. In certain embodiments, the at least one amino acid substitution is relative to SEQ ID NO: 1, provided herein. In certain embodiments, the variant IL-12 p40 subunit polypeptides have at least 70%, 80%, 85%, 90%, or 95% sequence identity to SEQ ID NO: 1. In certain embodiments, the variant IL-12 p40 subunit polypeptides have reduced affinity for IL-12 receptor subunit beta-1 (IL-12RB1). In certain embodiments, the at least one amino acid substitution is on an IL-12RB1 receptor contacting surface of the variant IL-12 p40 subunit polypeptide. In certain embodiments, the at least one amino acid substitution in the variant IL-12 p40 subunit polypeptide is located at an amino acid position that contacts IL-12RB1 through hydrogen bonds and/or ionic bonds. In certain embodiments, the at least one amino acid substitution in the variant IL-12 p40 subunit polypeptide is at a position that contacts IL-12RB1 through ionic bonds.

In another aspect, provided herein are variant IL-12 cytokines. In certain embodiments, the variant IL-12 cytokines comprise a variant IL-12 p40 subunit polypeptide described herein. In certain embodiments, the variant IL-12 cytokines comprise a variant IL-12 p40 subunit polypeptide described herein and a p35 subunit known in the art or as disclosed herein. Exemplary p35 subunits are described herein. In another aspect, provided herein are compositions comprising a variant IL-12 p40 subunit polypeptide or a variant IL-12 cytokine described herein. In certain embodiments, the compositions are pharmaceutical compositions. In certain embodiments, the compositions comprise the variant IL-12 p40 subunit or the variant IL-12 cytokine and one or more pharmaceutically acceptable diluents, excipients, or carriers. Also provided herein is a kit containing the variant IL-12 p40 subunit or variant IL-12 cytokine or composition of any of the foregoing embodiments, and instructions for use. In some embodiments, the variant IL-12 p40 subunit or variant IL-12 cytokine or composition is lyophilized. In some embodiments, the kit further includes a fluid for reconstitution of the lyophilized variant IL-12 p40 subunit or variant IL-12 cytokine.

In another aspect, provided herein are polynucleotides encoding the variant IL-12 p40 subunit polypeptides, p35 subunit polypeptides, variant p35 subunit polypeptides and/or variant IL-12 cytokines described herein. In a further aspect, provided herein are expression vectors comprising the polynucleotides. In a further aspect, provided herein are cells comprising the polynucleotides or expression vectors. In some embodiments, the cells are selected from a bacterial cells, a fungal cell and a mammalian cell. In some embodiments, the cells are selected from E. coli cells, Saccharomyces cerevisiae cells, and Chinese hamster ovary (CHO) cells.

In another aspect, provided herein are methods of treating, preventing, or diagnosing a disease or condition in a subject in need thereof, wherein the method includes administering to the subject an effective amount of the variant IL-12 p40 subunit or variant IL-12 cytokine of any of the foregoing embodiments, or a composition or a pharmaceutical composition containing the same. In some embodiments, the disease or condition is selected from a cancer, an autoimmune disease, an inflammatory disease, and an infection. In some embodiments, the effective amount is a therapeutically effective amount. In certain embodiments, the cancer is a solid tumor or a hematological malignancy.

Embodiments disclosed herein are also directed to the use of the variant IL-12 p40 subunit polypeptides or variant IL-12 cytokines of any of the foregoing embodiments for treating, preventing or diagnosing a disease or condition in a subject in need thereof. Embodiments disclosed herein are also directed the variant IL-12 p40 subunit polypeptides or variant IL-12 cytokines of any of the foregoing embodiments for use in treating, preventing, or diagnosing a disease or condition in a subject in need thereof. Embodiments disclosed herein are also directed the variant IL-12 p40 subunit polypeptides or variant IL-12 cytokines of any of the foregoing embodiments for use the manufacture of a medicament for treating, preventing, or diagnosing a disease or condition in a subject in need thereof. In some embodiments, the disease or condition is selected from a cancer, an autoimmune disease, an inflammatory disease, and an infection. In certain embodiments, the cancer is a solid tumor or a hematological malignancy.

These and other embodiments along with many of its features are described in more detail in conjunction with the text below.

### DETAILED DESCRIPTION OF THE EMBODIMENTS

Provided herein are variant IL-12 p40 subunit polypeptides and variant IL-12 cytokines and compositions comprising the same, wherein the variant IL-12 p40 subunit polypeptides comprise at least one amino acid substitution relative to a wild type IL-12 p40 subunit polypeptide. As disclosed herein, the at least one amino acid substitution at a specific site can improve the characteristics of the variant IL-12 p40 subunit polypeptide relative to a wild type (i.e., parent) IL-12 p40 subunit polypeptide. For example, amino acid substitutions as disclosed herein can lead to reduced receptor binding and/or reduced toxicity, relative to a wild type IL-12 p40 subunit polypeptide. This can lead to advantages with respect to the use of variant IL-12 p40 subunit polypeptides in therapy or diagnosis.

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The term “combinations thereof” includes every possible combination of elements to which the term refers to.

“Interleukin 12” or “IL-12” refers to an interleukin naturally produced in human cells in response to antigenic stimulation. IL-12 is a heterodimeric cytokine encoded by two separate genes. IL-12A encodes the IL-12 p35 subunit. Sequences include NM_000882, NM_001354582, NM_001354583, and NM_001397992 (mRNA); and NP_000873.2 (SEQ ID NO: 4), NP_001341511.1 (SEQ ID NO: 5), and NP_001341512.1 (SEQ ID NO: 6) (protein). A representative IL-12 p35 sequence is provided by SEQ ID NO: 3:

MRNLPVATPD PGMFPCLHHS QNLLRAVSNM
LQKARCTLEF YPCTSEEIDH EDITKDKTST
VEACLPLELT KNESCLNSRE TSFITNGSCL
ASRKTSEMMA LCLSSIYEDL KMYQVEFKTM
NAKLLMDPKR QIFLDQNMLA VIDELMQALN
FNSETVPQKS SLEEPDFYKT KIKLCILLHA
FRIRAVTIDR VMSYLNAS

IL-12B encodes the IL-12 p40 subunit. Sequences include NM_002187 (mRNA) and NP_002178 or P29460 (SEQ ID NO: 1) (protein). The amino acid sequence of P29460 is provided by SEQ ID NO: 1:

MCHQQLVISW FSLVFLASPL VAIWELKKDV
YVVELDWYPD APGEMVVLTC DTPEEDGITW
ILDQSSEVLG SGKTLTIQVK EFGDAGQYTC
HKGGEVLSHS LLLLHKKEDG IWSTDILKDQ
KEPKNKTFLR CEAKNYSGRF TCWWLTTIST
DLTFSVKSSR GSSDPQGVTC GAATLSAERV
RGDNKEYEYS VECQEDSACP AAEESLPIEV
MVDAVHKLKY ENYTSSFFIR DIIKPDPPKN
LQLKPLKNSR QVEVSWEYPD TWSTPHSYFS
LTFCVQVQGK SKREKKDRVF TDKTSATVIC
RKNASISVRA QDRYYSSSWS EWASVPCS.

A representative IL-12 p40 sequence is provided by SEQ ID NO: 2, which includes an N-terminal methionine and the mature form of a representative IL-12 p40 sequence:

MIWELKKDVY VVELDWYPDA PGEMVVLTCD
TPEEDGITWT LDQSSEVLGS GKTLTIQVKE
FGDAGQYTCH KGGEVLSHSL LLLHKKEDGI
WSTDILKQQK EPKNKTFLRC EAKNYSGRFT
CWWLTTISTQ LTFSVKSSRG SSDPQGVTCG
AATLSAERVR GDNKEYEYSV ECQEDSACPA
AEESLPIEVM VDAVHKLKYE NYTSSFFIRD
IIKPDPPKNL QLKPLKNSRQ VEVSWEYPDT
WSTPHSYFSL TFSVQVQGKS KREKKDRVFT
DKTSATVICR KNASISVRAQ DRYYSSSWSE
WASVPCS

SEQ ID NO: 7 provides the sequence of SEQ ID NO: 2 with a C-terminal 6His tag linked to SEQ ID NO: 2 by a GGGS linker.

The term “wild-type” or “parent” refers to a naturally occurring gene or protein. These include a naturally occurring IL-12 p40 subunit gene or protein and a naturally occurring IL-12 cytokine (comprising p35 and p40 subunits).

The term “variant” refers to a gene or protein encoding or having one or more amino acid additions, deletions, or substitutions compared to a parent gene or protein.

An “isolated variant IL-12 p40 subunit polypeptide” or “isolated variant IL-12 cytokine” is one that has been separated and/or recovered from a component of its natural environment. Components of the natural environment may include enzymes, hormones, and other proteinaceous or nonproteinaceous materials. In some embodiments, an isolated variant IL-12 p40 subunit polypeptide or variant IL-12 cytokine is purified to a degree sufficient to obtain at least 15 residues of N-terminal or internal amino acid sequence, for example by use of a spinning cup sequenator. In some embodiments, an isolated variant IL-12 p40 subunit polypeptide or variant IL-12 cytokine is purified to homogeneity by gel electrophoresis (e.g., SDS-PAGE) under reducing or nonreducing conditions, with detection by Coomassie blue or silver stain. In some aspects, an isolated variant IL-12 p40 subunit polypeptide or variant IL-12 cytokine is prepared by at least one purification step. The term can refer to the p40 subunit or to the cytokine, where appropriate.

The term “substantially pure” with respect to a composition comprising a variant IL-12 p40 subunit polypeptide or variant IL-12 cytokine refers to a composition that includes at least 80%, 85%, 90% or 95% by weight or, in certain embodiments, 95%, 98%, 99% or 100% by weight, e.g., dry weight, of the variant IL-12 p40 subunit polypeptide or variant IL-12 cytokine relative to the remaining portion of the composition. The weight percentage can be relative to the total weight of protein in the composition or relative to the total weight of variant IL-12 p40 subunit polypeptide or variant IL-12 cytokine in the composition. Purity can be determined by techniques apparent to those of skill in the art, for instance SDS-PAGE.

In some embodiments, an isolated variant IL-12 p40 subunit polypeptide or variant IL-12 cytokine is purified to at least 80%, 85%, 90%, 95%, or 99% by weight. In some embodiments, an isolated variant IL-12 p40 subunit polypeptide or variant IL-12 cytokine is purified to at least 80%, 85%, 90%, 95%, or 99% by volume. In some embodiments, an isolated variant IL-12 p40 subunit polypeptide or variant IL-12 cytokine is provided as a solution comprising at least 85%, 90%, 95%, 98%, 99% to 100% by weight. In some embodiments, an isolated variant IL-12 p40 subunit polypeptide or variant IL-12 cytokine is provided as a solution comprising at least 85%, 90%, 95%, 98%, 99% to 100% by volume.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 1-3 are, in some embodiments, considered conservative substitutions for one another.

TABLE 1
Selected groups of amino acids that are
considered conservative substitutions for
one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 2
Additional selected groups of amino acids
that are considered conservative substitutions
for one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 3
Further selected groups of amino acids that
are considered conservative substitutions
for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2^{nd} ed. (1993) W. H. Freeman & Co., New York, NY. An variant IL-12 p40 subunit polypeptide generated by making one or more conservative substitutions of amino acid residues in a parent IL-12 p40 subunit polypeptide is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of a variant IL-12 p40 subunit polypeptide or variant IL-12 cytokine or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a cancer, an inflammatory disease or condition, or an autoimmune disease or condition, that can be treated or diagnosed with a variant IL-12 p40 subunit polypeptide or variant IL-12 cytokine provided herein. In some embodiments, the subject is a human that has or is suspected to have cancer, an inflammatory disease or condition, or an autoimmune disease or condition.

### Variant IL-12 p40 Subunit Polypeptides

Provided herein are variant IL-12 p40 subunit polypeptides that comprise at least one amino acid substitution compared to a wild type IL-12 p40 subunit polypeptide. In some embodiments, the variant IL-12 p40 subunit polypeptides comprise at least two amino acid substitutions. In some embodiments, the variant IL-12 p40 subunit polypeptides comprise at least three, four, five, six, or more amino acid substitutions.

The at least one amino acid substitution can be made by standard techniques. In certain embodiments, the substitution is made by one or more mutations in the genetic sequence encoding the variant IL-12 p40 subunit polypeptides. It should be understood that throughout this disclosure, the position for amino acid substitutions in the variant IL-12 p40 subunit polypeptides is indicated with reference to the amino acid numbering of SEQ ID NO: 2 which is the mature form of SEQ ID NO: 1 with an N-terminal methionine. The corresponding positions of SEQ ID NO: 1 versus SEQ ID NO: 2 are +21, for example, position 16 of SEQ ID NO: 2 corresponds to position 37 of SEQ ID NO: 1.

In some embodiments, a variant IL-12 p40 subunit polypeptide comprises at least one amino acid substitution selected from the group consisting of: W16X, Y17A, P21A, D42A, D42K, Q43A, S44A, S45A, E46A, E46K, K59A, K59E, K85E, E87A, E87K, D88A, D88K, 190A, W91A, D94X, K103A, K103E, N104A, K105A, K105E, D162A, D162K, H195A, H195E, K196A, K196E, L197A, K198E, N201A, T203A, C253X, K259Y, K265Y, 1278Y, and combinations thereof, wherein X indicates any amino acid other than the SEQ ID NO: 1 amino acid at the indicated position. In some embodiments, a variant IL-12 p40 subunit polypeptide comprises two of the amino acid substitutions. In some embodiments, a variant IL-12 p40 subunit polypeptide comprises three of the amino acid substitutions. In some embodiments, a variant IL-12 p40 subunit polypeptide comprises four of the amino acid substitutions. In some embodiments, a variant IL-12 p40 subunit polypeptide comprises more than four of the amino acid substitutions. In certain embodiments, the variant IL-12 p40 subunit polypeptide further comprises a C253A amino acid substitution. In some embodiments, the amino acid substitution is relative to SEQ ID NO: 1.

In some embodiments, a variant IL-12 p40 subunit polypeptide comprises a W16X amino acid substitution, wherein X indicates any amino acid other than the SEQ ID NO: 1 amino acid at the indicated position. In some embodiments, a variant IL-12 p40 subunit polypeptide comprises a Y17A amino acid substitution. In some embodiments, a variant IL-12 p40 subunit polypeptide comprises a P21A amino acid substitution. In some embodiments, a variant IL-12 p40 subunit polypeptide comprises a D42A amino acid substitution. In some embodiments, a variant IL-12 p40 subunit polypeptide comprises a D42K amino acid substitution. In some embodiments, a variant IL-12 p40 subunit polypeptide comprises a Q43A amino acid substitution. In some embodiments, a variant IL-12 p40 subunit polypeptide comprises a S44A amino acid substitution. In some embodiments, a variant IL-12 p40 subunit polypeptide comprises a S45A amino acid substitution. In some embodiments, a variant IL-12 p40 subunit polypeptide comprises an E46A amino acid substitution. In some embodiments, a variant IL-12 p40 subunit polypeptide comprises a E46K amino acid substitution. In some embodiments, a variant IL-12 p40 subunit polypeptide comprises a K59A amino acid substitution. In some embodiments, a variant IL-12 p40 subunit polypeptide comprises a K59E amino acid substitution. In some embodiments, a variant IL-12 p40 subunit polypeptide comprises a K85E amino acid substitution. In some embodiments, a variant IL-12 p40 subunit polypeptide comprises an E87A amino acid substitution. In some embodiments, a variant IL-12 p40 subunit polypeptide comprises an E87K amino acid substitution. In some embodiments, a variant IL-12 p40 subunit polypeptide comprises a D88A amino acid substitution. In some embodiments, a variant IL-12 p40 subunit polypeptide comprises a D88K amino acid substitution. In some embodiments, a variant IL-12 p40 subunit polypeptide comprises a 190A amino acid substitution. In some embodiments, a variant IL-12 p40 subunit polypeptide comprises a W91A amino acid substitution. In some embodiments, a variant IL-12 p40 subunit polypeptide comprises a D94X amino acid substitution, wherein X indicates any amino acid other than the SEQ ID NO: 1 amino acid at the indicated position. In some embodiments, a variant IL-12 p40 subunit polypeptide comprises a K103A amino acid substitution. In some embodiments, a variant IL-12 p40 subunit polypeptide comprises a K103E amino acid substitution. In some embodiments, a variant IL-12 p40 subunit polypeptide comprises a N104A amino acid substitution. In some embodiments, a variant IL-12 p40 subunit polypeptide comprises a K105A amino acid substitution. In some embodiments, a variant IL-12 p40 subunit polypeptide comprises a K105E amino acid substitution. In some embodiments, a variant IL-12 p40 subunit polypeptide comprises a D162A amino acid substitution. In some embodiments, a variant IL-12 p40 subunit polypeptide comprises a D162K amino acid substitution. In some embodiments, a variant IL-12 p40 subunit polypeptide comprises a H195A amino acid substitution. In some embodiments, a variant IL-12 p40 subunit polypeptide comprises a H195E amino acid substitution. In some embodiments, a variant IL-12 p40 subunit polypeptide comprises a K196A amino acid substitution. In some embodiments, a variant IL-12 p40 subunit polypeptide comprises a K196E amino acid substitution. In some embodiments, a variant IL-12 p40 subunit polypeptide comprises a L197A amino acid substitution. In some embodiments, a variant IL-12 p40 subunit polypeptide comprises a K198E amino acid substitution. In some embodiments, a variant IL-12 p40 subunit polypeptide comprises a N201A amino acid substitution. In some embodiments, a variant IL-12 p40 subunit polypeptide comprises a T203A amino acid substitution. In some embodiments, a variant IL-12 p40 subunit polypeptide comprises a K259Y amino acid substitution. In some embodiments, a variant IL-12 p40 subunit polypeptide comprises a K265Y amino acid substitution. In some embodiments, a variant IL-12 p40 subunit polypeptide comprises an I278Y amino acid substitution. In certain embodiments, the variant IL-12 p40 subunit polypeptide further comprises a C253X amino acid substitution, wherein X indicates any amino acid other than the SEQ ID NO: 1 amino acid at the indicated position. In some embodiments, the amino acid substitution is relative to SEQ ID NO: 1.

In some embodiments, the variant IL-12 p40 subunit polypeptide comprises at least one amino acid substitution selected from the group consisting of: W16X, D94X, and K196E, wherein X indicates any amino acid other than the SEQ ID NO: 1 amino acid at the indicated position. In some embodiments, a variant IL-12 p40 subunit polypeptide comprises two of the amino acid substitutions. In some embodiments, a variant IL-12 p40 subunit polypeptide comprises three of the amino acid substitutions. In some embodiments, a variant IL-12 p40 subunit polypeptide comprises a W16X amino acid substitution, wherein X indicates any amino acid other than the SEQ ID NO: 1 amino acid at the indicated position. In some embodiments, a variant IL-12 p40 subunit polypeptide comprises a D94X amino acid substitution, wherein X indicates any amino acid other than the SEQ ID NO: 1 amino acid at the indicated position. In some embodiments, a variant IL-12 p40 subunit polypeptide comprises a K196E amino acid substitution. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitution is relative to SEQ ID NO: 1.

In some embodiments, the variant IL-12 p40 subunit polypeptide comprises less than five amino acid substitutions selected from the group consisting of: W16A, D94K, K105E, K196E, L197A, K259Y, and K265Y

In some embodiments, a variant IL-12 p40 subunit polypeptide comprises at least one amino acid substitution selected from the group consisting of: W16A, Y17A, P21A, D42A, D42K, Q43A, S44A, S45A, E46A, E46K, K59A, K59E, K85E, E87A, E87K, D88A, D88K, 190A, W91A, D94A, D94K, K103A, K103E, N104A, K105A, K105E, D162A, D162K, H195A, H195E, K196A, K196E, L197A, K198E, N201A, T203A, C253A, C253S, K259Y, K265Y, I278Y, and combinations thereof. In some embodiments, a variant IL-12 p40 subunit polypeptide comprises two of the amino acid substitutions. In some embodiments, a variant IL-12 p40 subunit polypeptide comprises three of the amino acid substitutions. In some embodiments, a variant IL-12 p40 subunit polypeptide comprises more than three of the amino acid substitutions. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitution is relative to SEQ ID NO: 1.

In some embodiments, a variant IL-12 p40 subunit polypeptide comprises amino acid substitutions selected from the group consisting of: W16A/D94K; W16A/K196E; D94K/K196E; W16A/D94K/K196E; W16A/D94K/K196E/L197A; W16A/D94K/K196E/K105E; W16A/D94K/K196E/K259Y; W16A/D94K/K196E/K265Y; K105E/K259Y; K105E/K265Y; K259Y/K265Y; and K105E/K259Y/K265Y, and combinations thereof. In some embodiments, a variant IL-12 p40 subunit polypeptide comprises W16A/D94K amino acid substitutions. In some embodiments, a variant IL-12 p40 subunit polypeptide comprises W16A/K196E amino acid substitutions. In some embodiments, a variant IL-12 p40 subunit polypeptide comprises W16A/D94K amino acid substitutions. In some embodiments, a variant IL-12 p40 subunit polypeptide comprises W16A/K196E amino acid substitutions. In some embodiments, a variant IL-12 p40 subunit polypeptide comprises D94K/K196E amino acid substitutions. In some embodiments, a variant IL-12 p40 subunit polypeptide comprises W16A/D94K/K196E amino acid substitutions. In some embodiments, a variant IL-12 p40 subunit polypeptide comprises W16A/D94K/K196E/L197A amino acid substitutions. In some embodiments, a variant IL-12 p40 subunit polypeptide comprises W16A/D94K/K196E/K105E amino acid substitutions. In some embodiments, variant p40 a IL-12 subunit polypeptide comprises W16A/D94K/K196E/K259Y amino acid substitutions. In some embodiments, a variant IL-12 p40 subunit polypeptide comprises W16A/D94K/K196E/K265Y amino acid substitutions. In some embodiments, a variant IL-12 p40 subunit polypeptide comprises K105E/K259Y amino acid substitutions. In some embodiments, a variant IL-12 p40 subunit polypeptide comprises K105E/K265Y amino acid substitutions. In some embodiments, a variant IL-12 p40 subunit polypeptide comprises K259Y/K265Y amino acid substitutions. In some embodiments, a variant IL-12 p40 subunit polypeptide comprises K105E/K259Y/K265Y amino acid substitutions. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises amino acid substitutions selected from the group consisting of: E87K/K196E, E87K/K198E, K196E/K198E and E87K/K196E/K198E, and combinations thereof. In some embodiments, a variant IL-12 p40 subunit polypeptide comprises E87K/K196E amino acid substitutions. In some embodiments, a variant IL-12 p40 subunit polypeptide comprises K196E/K198E amino acid substitutions. In some embodiments, a variant IL-12 p40 subunit polypeptide comprises E87K/K196E/K198E amino acid substitutions. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: W16A, K59E, K85E, E87K, D94A, D94K, K105E, K196A, K196E, L197A, K198E, K259Y, K265Y, E87K/K196E, E87K/K198E, K196E/K198E and E87K/K196E/K198E, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: W16A, D94K, K105E, K196E, L197A, K259Y, and K265Y, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: W16A, D94K, and K196E, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: W16A, D94K, K105E, K196E, L197A, K259Y, and K265Y, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: W16A, K59E, D94A, D94K, K196A, K196E, L197A, K198E, E87K, K196E, K198E, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises at least one amino acid substitution selected from the group consisting of: E87K, K196E, K198E, and combinations thereof. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, a variant IL-12 p40 subunit polypeptide comprises amino acid substitutions selected from the group consisting of: E87K/K196E, E87K/K198E, K196E/K198E and E87K/K196E/K198E. In certain embodiments, the variant IL-12 p40 subunit polypeptide further comprises a C253A amino acid substitution. In certain embodiments, the variant IL-12 p40 subunit polypeptide further comprises a C253S amino acid substitution. In some embodiments, the amino acid substitutions are relative to SEQ ID NO: 1.

In some embodiments, the variant IL-12 p40 subunit polypeptide has at least 70% sequence identity to SEQ ID NO: 1. In some embodiments, the variant IL-12 p40 subunit polypeptide has at least 75% sequence identity to SEQ ID NO: 1. In some embodiments, the variant IL-12 p40 subunit polypeptide has at least 80% sequence identity to SEQ ID NO: 1. In some embodiments, the variant IL-12 p40 subunit polypeptide has at least 85% sequence identity to SEQ ID NO: 1. In some embodiments, the variant IL-12 p40 subunit polypeptide has at least 90% sequence identity to SEQ ID NO: 1. In some embodiments, the variant IL-12 p40 subunit polypeptide has at least 95% sequence identity to SEQ ID NO: 1. In some embodiments, the variant IL-12 p40 subunit polypeptide has at least 96% sequence identity to SEQ ID NO: 1. In some embodiments, the variant IL-12 p40 subunit polypeptide has at least 97% sequence identity to SEQ ID NO: 1. In some embodiments, the variant IL-12 p40 subunit polypeptide has at least 98% sequence identity to SEQ ID NO: 1. In some embodiments, the variant IL-12 p40 subunit polypeptide has at least 99% sequence identity to SEQ ID NO: 1. In certain embodiments, the variant IL-12 p40 subunit polypeptide lacks an N-terminal methionine.

In some embodiments, the variant IL-12 p40 subunit polypeptide has at least 70% sequence identity to SEQ ID NO: 2. In some embodiments, the variant IL-12 p40 subunit polypeptide has at least 75% sequence identity to SEQ ID NO: 2. In some embodiments, the variant IL-12 p40 subunit polypeptide has at least 80% sequence identity to SEQ ID NO: 2. In some embodiments, the variant IL-12 p40 subunit polypeptide has at least 85% sequence identity to SEQ ID NO: 2. In some embodiments, the variant IL-12 p40 subunit polypeptide has at least 90% sequence identity to SEQ ID NO: 2. In some embodiments, the variant IL-12 p40 subunit polypeptide has at least 95% sequence identity to SEQ ID NO: 2. In some embodiments, the variant IL-12 p40 subunit polypeptide has at least 96% sequence identity to SEQ ID NO: 2. In some embodiments, the variant IL-12 p40 subunit polypeptide has at least 97% sequence identity to SEQ ID NO: 2. In some embodiments, the variant IL-12 p40 subunit polypeptide has at least 98% sequence identity to SEQ ID NO: 2. In some embodiments, the variant IL-12 p40 subunit polypeptide has at least 99% sequence identity to SEQ ID NO: 2. In certain embodiments, the variant IL-12 p40 subunit polypeptide lacks an N-terminal methionine.

Also within the scope are post-translationally modified variants of the variant IL-12 p40 subunit polypeptides disclosed herein. Any of the variant IL-12 p40 subunit polypeptides provided herein can be post-translationally modified in any manner recognized by those of skill in the art. Typical post-translational modifications for variant IL-12 p40 subunit polypeptides include interchain disulfide bonding and glycosylation. The post-translational modification can occur during production, in vivo, in vitro or otherwise. In some embodiments, the post-translational modification can be an intentional modification by a practitioner, for instance, using the methods provided herein.

Further included within the scope are variant IL-12 p40 subunit polypeptides fused to further peptides or polypeptides. Exemplary fusions include, but are not limited to, e.g., a methionyl variant IL-12 p40 subunit polypeptide in which a methionine is linked to the N-terminus of the variant IL-12 p40 subunit polypeptide resulting from recombinant expression, fusions for the purpose of purification (including but not limited to, to poly-histidine or affinity epitopes), fusions with serum albumin binding peptides, and fusions with serum proteins such as serum albumin. The variant IL-12 p40 subunit polypeptides may comprise protease cleavage sequences, variant IL-12 p40 subunit-binding domains (including but not limited to, FLAG or poly-His) or other affinity based sequences (including but not limited to, FLAG, poly-His, GST, etc.). The variant IL-12 p40 subunit polypeptides may also comprise linked molecules that improve detection (including but not limited to, GFP), purification, or other features of the variant IL-12 p40 subunit polypeptide. In certain embodiments, the variant IL-12 p40 subunit polypeptides comprise a C-terminal affinity sequence that facilitates purification of full length variant IL-12 p40 subunit polypeptides. In certain embodiments, such C-terminal affinity sequence is a poly-His sequence, e.g., a 6-His sequence.

A variant IL-12 cytokine typically comprises multiple polypeptide chains. In certain embodiments, provided herein is a variant IL-12 cytokine comprising the variant IL-12 p40 subunit polypeptide described herein and an IL-12 p35 subunit polypeptide or a variant IL-12 p35 subunit polypeptide. The IL-12 p35 subunit polypeptide or variant IL-12 p35 subunit polypeptide can be any p35 subunit or variant thereof known to the person of skill. In certain embodiments, the IL-12 p35 subunit has a sequence selected from SEQ ID NO: 3, NP_000873 (SEQ ID NO: 4), NP_001341511 (SEQ ID NO: 5), and NP_001341512 (SEQ ID NO: 6). In certain embodiments, the IL-12 p35 subunit polypeptide or variant IL-12 p35 subunit polypeptide can have at least 70%, 80%, 85%, 90%, or 95% sequence identity to SEQ ID NO: 3. In certain embodiments, the IL-12 p35 subunit polypeptide or variant IL-12 p35 subunit polypeptide can have at least 70%, 80%, 85%, 90%, or 95% sequence identity to SEQ ID NO: 4. In certain embodiments, the IL-12 p35 subunit polypeptide or variant IL-12 p35 subunit polypeptide can have at least 70%, 80%, 85%, 90%, or 95% sequence identity to SEQ ID NO: 5. In certain embodiments, the IL-12 p35 subunit polypeptide or variant IL-12 p35 subunit polypeptide can have at least 70%, 80%, 85%, 90%, or 95% sequence identity to SEQ ID NO: 6. In certain embodiments, the IL-12 p35 subunit polypeptide or variant IL-12 p35 subunit polypeptide lacks an N-terminal methionine.

It should be understood that throughout this disclosure, the position for amino acid substitutions in the variant IL-12 p35 subunit polypeptides is indicated with reference to the amino acid numbering of SEQ ID NO: 3 which is the mature form of the IL-12 p35 subunit with an N-terminal methionine. The corresponding positions of SEQ ID NOs: 4-6 can be determined by alignment of the sequence of SEQ ID NO: 3 beginning with amino acid position 2 with SEQ ID NOs: 4-6.

In some embodiments, the variant IL-12 p35 subunit polypeptide comprises at least one amino acid substitution selected from the group consisting of: C16X and C89X, wherein X indicates any amino acid other than the SEQ ID NO: 3 amino acid at the indicated position. In some embodiments, a variant IL-12 p35 subunit polypeptide comprises two of the amino acid substitutions. In some embodiments, a variant IL-12 p35 subunit polypeptide comprises a C16X substitution, wherein X indicates any amino acid other than the SEQ ID NO: 3 amino acid at the indicated position. In some embodiments, a variant IL-12 p35 subunit polypeptide comprises a C16S substitution. In some embodiments, a variant IL-12 p35 subunit polypeptide comprises a C89X substitution, wherein X indicates any amino acid other than the SEQ ID NO: 1 amino acid at the indicated position. In some embodiments, a variant IL-12 p35 subunit polypeptide comprises a C89S substitution. In some embodiments, the amino acid substitution is relative to SEQ ID NO: 3.

In certain embodiments, provided herein is a dimer comprising a variant IL-12 p40 subunit polypeptide described herein. In certain embodiments, provided herein is a dimer comprising two variant IL-12 p40 subunit polypeptides described herein.

In certain embodiments, the variant IL-12 p40 subunit polypeptide has reduced affinity for IL-12 receptor subunit beta-1 (IL-12Rβ1). In certain embodiments, the at least one amino acid substitution is on an IL-12Rβ1 receptor contacting surface of the variant IL-12 p40 subunit polypeptide. In certain embodiments, the at least one amino acid substitution in the variant IL-12 p40 subunit polypeptide is located at an amino acid position that contacts IL-12Rβ1 through hydrogen bonds and/or ionic bonds. In certain embodiments, the at least one amino acid substitution in the variant IL-12 p40 subunit polypeptide is at a position that contacts IL-12Rβ1 through ionic bonds. In some embodiments, the variant IL-12 cytokine has reduced affinity for IL-12 receptor subunit beta-1 (IL-12Rβ1).

In some embodiments, the at least one amino acid substitution provides a variant IL-12 p40 subunit that has reduced receptor binding. In some embodiments, the at least one amino acid substitution provides a variant IL-12 p40 subunit polypeptide that has reduced toxicity. In some embodiments, the at least one amino acid substitution provides a variant IL-12 p40 subunit polypeptide that has reduced receptor binding and reduced toxicity. In some embodiments, the variant IL-12 cytokine has reduced receptor binding.

In certain embodiments, the at least one amino acid substitution reduce binding of the variant IL-12 p40 subunit polypeptide to IL-12Rβ1. In certain embodiments, the at least one amino acid substitution reduces binding of the variant IL-12 p40 subunit polypeptide to IL-12Rβ1 relative to wild-type IL-12 p40. In certain embodiments, the at least one amino acid substitution reduces binding of the variant IL-12 p40 subunit polypeptide to IL-12Rβ1 relative to an IL-12 p40 subunit polypeptide of the same sequence, other than the at least one amino acid substitution. In certain embodiments, the at least one amino acid substitution reduces binding of variant IL-12 p40 subunit polypeptide to IL-12Rβ1 by 10%, 20%, 25%, 50%, 75%, 100%, 125%, 150%, 200%, 250%, 300%, 400%, 500%, 1000%, 2000%, 3000%, or more.

In certain embodiments, the at least one amino acid substitution reduces the stability of the variant IL-12 p40 subunit polypeptide. In certain embodiments, the at least one amino acid substitution reduces the serum half-life of the variant IL-12 p40 subunit polypeptide. In certain embodiments, the at least one amino acid substitution reduces the serum half-life of the variant IL-12 p40 subunit polypeptide relative to wild-type IL-12 p40. In certain embodiments, the at least one amino acid substitution reduces the serum half-life of the variant IL-12 p40 subunit polypeptide relative to an IL-12 p40 of the same sequence, other than the at least one amino acid substitution. In certain embodiments, the at least one amino acid substitution reduce the serum half-life of the variant IL-12 p40 subunit polypeptide by 10%, 20%, 25%, 50%, 75%, 100%, 125%, 150%, 200%, 250%, 300%, 400%, 500%, 1000%, 2000%, 3000%, or more.

In certain embodiments, the at least one amino acid substitution reduces binding of the variant IL-12 cytokine to IL-12Rβ1. In certain embodiments, the at least one amino acid substitution reduces binding of the variant IL-12 cytokine to IL-12Rβ1 relative to wild-type IL-12. In certain embodiments, the at least one amino acid substitution reduces binding of the variant IL-12 cytokine to IL-12Rβ1 relative to an IL-12 of the same sequence, other than the at least one amino acid substitution. In certain embodiments, the at least one amino acid substitution reduces binding of the variant IL-12 cytokine to IL-12RB1 by 10%, 20%, 25%, 50%, 75%, 100%, 125%, 150%, 200%, 250%, 300%, 400%, 500%, 1000%, 2000%, 3000%, or more.

In certain embodiments, the at least one amino acid substitution reduces the stability of the variant IL-12 cytokine. In certain embodiments, the at least one amino acid substitution reduces the serum half-life of the variant IL-12 cytokine. In certain embodiments, the at least one amino acid substitution reduces the serum half-life of the variant IL-12 cytokine relative to wild-type IL-12. In certain embodiments, the at least one amino acid substitution reduces the serum half-life of the variant IL-12 cytokine relative to an IL-12 of the same sequence, other than the at least one amino acid substitution. In certain embodiments, the at least one amino acid substitution reduces the serum half-life of the variant IL-12 cytokine by 10%, 20%, 25%, 50%, 75%, 100%, 125%, 150%, 200%, 250%, 300%, 400%, 500%, 1000%, 2000%, 3000%, or more.

### Vectors, Host Cells, and Recombinant Methods

Also provided are isolated nucleic acids encoding variant IL-12 p40 subunit polypeptides, p35 subunit polypeptides, variant p35 subunit polypeptides and/or variant IL-12 cytokines, vectors and host cells comprising the nucleic acids, and recombinant techniques for the production of the variant IL-12 p40 subunit polypeptides and variant IL-12 cytokines.

For recombinant production of the variant IL-12 p40 subunit polypeptides, p35 subunit polypeptides, variant p35 subunit polypeptides and/or variant IL-12 cytokines, the nucleic acid encoding it may be isolated and inserted into a replicable vector for further cloning (i.e., amplification of the DNA) or expression. In some aspects, the nucleic acid may be produced by homologous recombination, for example as described in U.S. Pat. No. 5,204,244.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615.

Illustrative examples of suitable host cells are provided below. these host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for variant IL-12 p40 subunit polypeptide-, p35 subunit polypeptide-, variant p35 subunit polypeptide- and/or variant IL-12 cytokine-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K. thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the variant IL-12 p40 subunit polypeptides, p35 subunit polypeptides, variant p35 subunit polypeptides and/or variant IL-12 cytokines may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the variant IL-12 p40 subunit polypeptides, p35 subunit polypeptides, variant p35 subunit polypeptides and/or variant IL-12 cytokines can be produced intracellularly, in the periplasmic space, or directly secreted into the medium. If the variant IL-12 p40 subunit polypeptide is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating polypeptides which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation.

In some embodiments, the variant IL-12 p40 subunit polypeptide, p35 subunit polypeptide, variant p35 subunit polypeptide and/or variant IL-12 cytokine is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the variant IL-12 p40 subunit polypeptide, p35 subunit polypeptide, variant p35 subunit polypeptide and/or variant IL-12 cytokine may be useful, for example, where the variant IL-12 p40 subunit polypeptide, p35 subunit polypeptide, variant p35 subunit polypeptide and/or variant IL-12 cytokine accumulates in a cell as an insoluble aggregate, or where yields from periplasmic expression are low.

Where the variant IL-12 p40 subunit polypeptide, p35 subunit polypeptide, variant p35 subunit polypeptide and/or variant IL-12 cytokine is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The variant IL-12 p40 subunit polypeptide, p35 subunit polypeptide, variant p35 subunit polypeptide and/or variant IL-12 cytokine composition prepared from the cells can be purified using, for example, hydroxylapatite chromatography, gel electrophoresis, dialysis, and affinity chromatography, with affinity chromatography being a particularly useful purification technique.

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art.

Following any preliminary purification step(s), the mixture comprising the variant IL-12 p40 subunit polypeptide of interest and contaminants may be subjected to low pH hydrophobic interaction chromatography using an elution buffer at a pH between about 2.5-4.5, generally performed at low salt concentrations (e.g., from about 0-0.25 M salt).

### Pharmaceutical Compositions and Methods of Administration

The variant IL-12 p40 subunit polypeptides, p35 subunit polypeptides, variant p35 subunit polypeptides or variant IL-12 cytokines provided herein can be formulated into pharmaceutical compositions using methods available in the art and those disclosed herein. Any of the variant IL-12 p40 subunit polypeptides, p35 subunit polypeptides, variant p35 subunit polypeptides or variant IL-12 cytokines provided herein can be provided in the appropriate pharmaceutical composition and be administered by a suitable route of administration.

The methods provided herein encompass administering pharmaceutical compositions comprising at least one variant IL-12 p40 subunit polypeptide or variant IL-12 cytokine provided herein and one or more compatible and pharmaceutically acceptable carriers. In this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E. W., Remington's Pharmaceutical Sciences.

In clinical practice the pharmaceutical compositions or variant IL-12 p40 subunit polypeptides or variant IL-12 cytokines provided herein may be administered by any route known in the art. In certain embodiments, a pharmaceutical composition or variant IL-12 p40 subunit polypeptide or variant IL-12 cytokine provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing and stabilizing agents. Sterilization can be carried out in several ways, for example using a bacteriological filter, by radiation or by heating. Parenteral compositions can also be prepared in the form of sterile solid compositions which can be dissolved at the time of use in sterile water or any other injectable sterile medium.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic variant IL-12 p40 subunit polypeptides or variant IL-12 cytokines.

Typical pharmaceutical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a pharmaceutical composition or dosage form depends on a variety of factors well known in the art including, but not limited to, the way in which the dosage form will be administered to a subject and the specific variant IL-12 p40 subunit polypeptide or variant IL-12 cytokine in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmocopeia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, variant IL-12 p40 subunit polypeptides or variant IL-12 cytokines are supplied as a water free concentrate.

In another embodiment, the pharmaceutical composition is supplied in liquid form. In some embodiments, the pharmaceutical composition is provided in liquid form and is substantially free of surfactants and/or inorganic salts.

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Further encompassed herein are anhydrous pharmaceutical compositions and dosage forms comprising a variant IL-12 p40 subunit polypeptide or variant IL-12 cytokine, since water can facilitate the degradation of some variant IL-12 p40 subunit polypeptides or variant IL-12 cytokines.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Further provided are pharmaceutical compositions and dosage forms that comprise one or more excipients that reduce the rate by which a variant IL-12 p40 subunit polypeptide or variant IL-12 cytokine will decompose. Such excipients, which are referred to herein as “stabilizers,” include, but are not limited to, antioxidants such as ascorbic acid, pH buffers, or salt buffers.

### Parenteral Dosage Forms

In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the variant IL-12 p40 subunits disclosed herein can also be incorporated into the parenteral dosage forms.

### Dosage and Unit Dosage Forms

In human therapeutics, the doctor will determine the posology which he considers most appropriate according to a preventive or curative treatment and according to the age, weight, stage of the infection and other factors specific to the subject to be treated.

The amount of the variant IL-12 p40 subunit polypeptide or variant IL-12 cytokine or composition which will be effective in the prevention or treatment of a disorder or one or more symptoms thereof will vary with the nature and severity of the disease or condition, and the route by which the variant IL-12 p40 subunit polypeptide or variant IL-12 cytokine or composition is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

The dose can be administered according to a suitable schedule, for example, once, two times, three times, or for times weekly. It may be necessary to use dosages of the variant IL-12 p40 subunit polypeptide or variant IL-12 cytokine outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the variant IL-12 p40 subunit polypeptide or variant IL-12 cytokine provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of a variant IL-12 p40 subunit polypeptide or variant IL-12 cytokine or composition provided herein followed by one or more maintenance doses.

In certain embodiments, a dose of a variant IL-12 p40 subunit polypeptide or variant IL-12 cytokine or composition provided herein can be administered to achieve a steady-state concentration of the variant IL-12 p40 subunit polypeptide or variant IL-12 cytokine in blood or serum of the subject. The steady-state concentration can be determined by measurement according to techniques available to those of skill or can be based on the physical characteristics of the subject such as height, weight and age.

### Therapeutic Applications

For therapeutic applications, variant IL-12 p40 subunit polypeptides or variant IL-12 cytokines disclosed herein are administered to a mammal, generally a human, in a pharmaceutically acceptable dosage form such as those known in the art and those discussed above. For example, the variant IL-12 p40 subunit polypeptides or variant IL-12 cytokines disclosed herein may be administered to a human intravenously as a bolus or by continuous infusion over a period of time, by systemic, intravenous, intramuscular, intraperitoneal, intra-cerebrospinal, subcutaneous, intra-articular, intrasynovial, intrathecal, or intratumoral routes. The variant IL-12 p40 subunit polypeptides or variant IL-12 cytokines also are suitably administered by peritumoral, intralesional, or perilesional routes, to exert local as well as systemic therapeutic effects.

A therapeutically effective amount of the variant IL-12 p40 subunit polypeptide or variant IL-12 cytokine or composition is an amount that is effective to reduce the severity, the duration and/or the symptoms of a particular disease or condition. The amount of the variant IL-12 p40 subunit polypeptide or variant IL-12 cytokine or composition that will be therapeutically effective in the prevention, management, treatment and/or amelioration of a particular disease can be determined by standard clinical techniques. The precise amount of the variant IL-12 p40 subunit polypeptide or variant IL-12 cytokine or composition to be administered with depend, in part, on the route of administration, the seriousness of the particular disease or condition, and should be decided according to the judgment of the practitioner and each subject's circumstances.

In certain embodiments, a method for treating a disease comprises administering a therapeutically effective amount of a variant IL-12 p40 subunit polypeptide, a variant IL-12 cytokine, or the composition thereof of the present disclosure to a subject in need thereof.

In certain embodiments, a method of inducing T cell expansion in a patient in need thereof comprises administering a therapeutically effective amount of a variant IL-2 p40 subunit polypeptide a variant IL-12 cytokine, or the composition thereof of the present disclosure to a subject in need thereof.

In certain embodiments, a method of treatment can further comprise administering a second therapeutic agent. In certain embodiments, the second therapeutic agent can be administered simultaneously with the variant IL-12 p40 subunit polypeptide, variant IL-12 cytokine or composition. In certain embodiments, the second therapeutic agent and the variant IL-12 p40 subunit polypeptide, variant IL-12 cytokine or composition can be administered sequentially.

In certain embodiments, the second therapeutic agent can be selected from the group consisting of a chemotherapeutic agent, one or more additional cytokines, a variant IL-12 p40 subunit polypeptide, a variant IL-12 cytokine, an antiangiogenic agent, radiotherapy, adoptive therapy, and a tumor vaccine.

In certain embodiments, the second therapeutic agent can be an immune checkpoint inhibitor. In certain embodiments, the immune checkpoint inhibitor can be selected from the group consisting of an anti-PD-1 variant IL-12 p40 subunit, an anti-PD-LI variant IL-12 p40 subunit, an anti-TIM3 variant IL-12 p40 subunit, an anti-TIGIT variant IL-12 p40 subunit, an anti-LAG3 variant IL-12 p40 subunit, and an anti-CTLA-4 variant IL-12 p40 subunit.

In certain embodiments, the disease can be a cancer. In certain embodiments, a composition of the present disclosure can be for use in treating cancer. In certain embodiments, the disease can be a solid or an hematological cancer.

### Diagnostic Applications

In some embodiments, the variant IL-12 p40 subunit polypeptides or variant IL-12 cytokines provided herein are used in diagnostic applications. For example, a variant IL-12 p40 subunit polypeptide or variant IL-12 cytokine disclosed herein that is specific for a given receptor may be useful in assays for the given receptor. In some aspects the variant IL-12 p40 subunit polypeptide or variant IL-12 cytokine can be used to detect the expression of the given receptor in various cells and tissues. These assays may be useful, for example, diagnosing cancer, infection and autoimmune disease.

In the methods, the formation of a complex between the variant IL-12 p40 subunit polypeptide or variant IL-12 cytokine and receptor can be detected by any method known to those of skill in the art. Examples include assays that use secondary reagents for detection, ELISA's and immunoprecipitation and agglutination assays. A detailed description of these assays is, for example, given in Harlow and Lane, Variant IL-12 p40 subunits: A Laboratory Manual (Cold Spring Harbor Laboratory, New York 1988 555-612, WO 96/13590 to Maertens and Stuyver, Zrein et al. (1998) and WO 96/29605.

For in situ diagnosis, the variant IL-12 p40 subunit polypeptide or variant IL-12 cytokine may be administered to a subject by methods known in the art such as, for example, intravenous, intranasal, intraperitoneal, intracerebral, intraarterial injection such that a specific binding between the variant IL-12 p40 subunit polypeptide or variant IL-12 cytokine and receptor may occur. The variant IL-12 p40 subunit polypeptide or variant IL-12 cytokine/receptor complex may conveniently be detected through a label attached to the variant IL-12 p40 subunit polypeptide or variant IL-12 cytokine or any other art-known method of detection.

In some diagnostic applications, the variant IL-12 p40 subunit polypeptide or variant IL-12 cytokine may be labeled with a detectable moiety. Suitable detectable moieties include, but are not limited to radioisotopes, fluorescent labels, and enzyme-substrate labels.

### Kits

In some embodiments, a variant IL-12 p40 subunit polypeptide or variant IL-12 cytokine as described herein can be provided in a kit, i.e., a packaged combination of reagents in predetermined amounts with instructions for performing a procedure. In some embodiments, the procedure is a diagnostic assay. In other embodiments, the procedure is a therapeutic procedure.

### EXAMPLES

### Example 1

### High-Throughput Cell-Free Expression of IL-12 Variants

IL-12p40_C253S was modified with a carboxy-terminal 6HIS-Tag sequence (IL-12p40-6HIS_C253S (SEQ ID NO: 8)) to facilitate purification of the variants. IL-12p35_C16S_C89S (SEQ ID NO: 9) was co-expressed at various DNA ratios with the IL-12p40-HIS6_C253S to identify the best IL-12p70-6HIS product yield. These variants were expressed in XpressCF+® in an overnight reaction in the presence of ¹⁴C-Leucine. The production of the IL-12p70-6HIS variants was estimated by ¹⁴C-incorporation (total yield), and the amount remaining in solution (soluble yield) was further measured following centrifugation at 14,000×g for 10 minutes.

Expression and recovery of variant constructs were also employed to determine optimal recovery and activity of IL-12p70-6HIS constructs. For each variant, the p40 subunit had the sequence of SEQ ID NO: 2 with the specified amino acid substitutions and a C-terminal 6His-tag, and the p35 subunit had the sequence of SEQ ID NO: 3 with the specified amino acid substitutions. The IL-12p70-6HIS variants were expressed in an XpressCF+® reaction. The cell-free extracts were prepared from a mixture of four extracts derived from the following two engineered strains described as: (1) an OmpT sensitive RF1 attenuated E. coli strain engineered to overexpress E. coli DsbC and FkpA, and (2) a similar RF1 attenuated E. coli strain engineered to produce T7 RNA polymerase. Cell-free extract (1) was treated with 50 μM iodoacetamide for 30 min at RT (20° C.) and added to a premix containing all other components. The final concentration in the protein synthesis reaction was 37% (v/v) cell extract (1), 0.5% (v/v) cell extract (2), 2 mM GSSG, 8 mM magnesium glutamate, 10 mM ammonium glutamate, 130 mM potassium glutamate, 35 mM sodium pyruvate, 1.2 mM AMP, 0.86 mM each of GMP, UMP, and CMP, 2 mM amino acids (except 1 mM for Tyrosine), 4 mM sodium oxalate, 1 mM putrescine, 1.5 mM spermidine, 15 mM potassium phosphate, 5 μg/mL IL-12p70-6HIS or variant DNA at ratios of 1 to 1-3 (p40 to p35).

Cell-free reactions were performed at 20° C. for 12 hours on a shaker at 650 rpm in 96-well plates at 100 μL scale or 48-cylinder well assay blocks at 1 mL scale. Other expression approaches include methods without stirring in 100×10 mm petri dish at 8 mL scale or in stirred tanks at larger scales. IL-12p70-6HIS variants were purified by immobilized metal affinity chromatography (IMAC) purification methods incorporating 2 washes with PBS pH 7.2 and elution in a PBS with 300 mM imidazole pH 7.2. After buffer exchange into low salt phosphate buffer, 50 mM sodium-phosphate pH 7.2, the samples were captured on equilibrated CaptoQ resin, washed 1× low salt buffer before elution in 50 mM phosphate with 200 mM NaCl pH 7.2. IL-12p70 and variant concentrations were determined by A280.

### Example 2

### Differential Scanning Fluorimetry (DSF) of IL-12 Variants

A protein thermal shift assay was carried out by mixing the protein to be assayed with an environmentally sensitive dye (SYPRO Orange, Life Technologies Cat #S-6650) in a phosphate buffered solution, monitoring the fluorescence of the mixture in real time as it underwent controlled thermal denaturation. Protein solutions between 0.05-2 mg/mL were mixed at a 1:1 volumetric ratio with a 500-fold phosphate buffered-diluted solution of SYPRO Orange (SYPRO Orange stock dye was 5000× in DMSO). 5 μL aliquots of the protein-dye mixture were dispensed in quadruplicate in a 384-well microplate (Bio-Rad Cat #MSP-3852, and the plate was sealed with an optically clear sealing film (Bio-Rad Cat #MSB-1001) and placed in a 384-well plate real-time thermocycler (Bio-Rad CFX384 Real Time System). The protein-dye mixture was heated from 25° C. to 95° C., at increments of 0.1° C. per cycle (about 1.5° C. per minute), allowing 3 seconds of equilibration at each temperature before taking a fluorescence measurement. At the end of the experiment, the transition melting temperature was determined using the Bio-Rad CFX manager software. Tables 4 and 5 provide the DSF results for certain IL-12p70 variants.

TABLE 4
IL-12p70 variants with steering mutations in IL-12p40:
Differential scanning fluorimetry results

| p40 Description | p35 Description | Tm, C |
|---|---|---|
| IL-12P40-HIS6_C253S | IL-12P35_C16S_C89S | 54.8 |
| IL-12P40-HIS6_C253S_W16A | IL-12P35_C16S_C89S | 55.2 |
| IL-12P40-HIS6_C253S_Y17A | IL-12P35_C16S_C89S | 55.5 |
| IL-12P40-HIS6_C253S_P21A | IL-12P35_C16S_C89S | 54.2 |
| IL-12P40-HIS6_C253S_D42A | IL-12P35_C16S_C89S | 54.2 |
| IL-12P40-HIS6_C253S_Q43A | IL-12P35_C16S_C89S | 54.6 |
| IL-12P40-HIS6_C253S_S44A | IL-12P35_C16S_C89S | 54.2 |
| IL-12P40-HIS6_C253S_S45A | IL-12P35_C16S_C89S | 54.8 |
| IL-12P40-HIS6_C253S_E46A | IL-12P35_C16S_C89S | 54.6 |
| IL-12P40-HIS6_C253S_K59A | IL-12P35_C16S_C89S | 53.2 |
| IL-12P40-HIS6_C253S_E87A | IL-12P35_C16S_C89S | 54.2 |
| IL-12P40-HIS6_C253S_D88A | IL-12P35_C16S_C89S | 54.6 |
| IL-12P40-HIS6_C253S_190A | IL-12P35_C16S_C89S | 55.2 |
| IL-12P40-HIS6_C253S_W91A | IL-12P35_C16S_C89S | 55.5 |
| IL-12P40-HIS6_C253S_D94A | IL-12P35_C16S_C89S | 53.1 |
| IL-12P40-HIS6_C253S_K103A | IL-12P35_C16S_C89S | 54.1 |
| IL-12P40-HIS6_C253S_N104A | IL-12P35_C16S_C89S | 55.6 |
| IL-12P40-HIS6_C253S_K105A | IL-12P35_C16S_C89S | 53.9 |
| IL-12P40-HIS6_C253S_D162A | IL-12P35_C16S_C89S | 54.6 |
| IL-12P40-HIS6_C253S_H195A | IL-12P35_C16S_C89S | 52 |
| IL-12P40-HIS6_C253S_K196A | IL-12P35_C16S_C89S | 54 |
| IL-12P40-HIS6_C253S_L197A | IL-12P35_C16S_C89S | 54.7 |
| IL-12P40-HIS6_C253S_N201A | IL-12P35_C16S_C89S | 54.5 |
| IL-12P40-HIS6_C253S_T203A | IL-12P35_C16S_C89S | 54.2 |
| IL-12P40-HIS6_C253S_D42K | IL-12P35_C16S_C89S | 52.8 |
| IL-12P40-HIS6_C253S_E46K | IL-12P35_C16S_C89S | 55 |
| IL-12P40-HIS6_C253S_K59E | IL-12P35_C16S_C89S | 51.9 |
| IL-12P40-HIS6_C253S_E87K | IL-12P35_C16S_C89S | 55.2 |
| IL-12P40-HIS6_C253S_D88K | IL-12P35_C16S_C89S | 55.1 |
| IL-12P40-HIS6_C253S_D94K | IL-12P35_C16S_C89S | 53.3 |
| IL-12P40-HIS6_C253S_K103E | IL-12P35_C16S_C89S | 53.8 |
| IL-12P40-HIS6_C253S_K105E | IL-12P35_C16S_C89S | 54.1 |
| IL-12P40-HIS6_C253S_D162K | IL-12P35_C16S_C89S | 54.5 |
| IL-12P40-HIS6_C253S_H195E | IL-12P35_C16S_C89S | 50.4 |
| IL-12P40-HIS6_C253S_K196E | IL-12P35_C16S_C89S | 53.8 |
| IL-12P40-HIS6_C253S_K259Y | IL-12P35_C16S_C89S | 54.8 |
| IL-12P40-HIS6_C253S_K265Y | IL-12P35_C16S_C89S | 55 |
| IL-12P40-HIS6_C253S_1278Y | IL-12P35_C16S_C89S | 54.9 |
| IL-12P40-HIS6_C253S_E87K, | IL-12P35_C16S_C89S | 54 |
| K196E | | |
| IL-12P40-HIS6_C253S_E87K, | IL-12P35_C16S_C89S | 52.2 |
| K196E, K198E | | |

TABLE 5
IL-12p70 variants with multiple steering mutations in IL-12p40:
Differential scanning fluorimetry results

| | | Tm, |
|---|---|---|
| p40 Description | p35 Description | º C. |
| IL-12P40-HIS6_C253S | IL-12P35_C16S_C89S | 55.1 |
| IL-12P40-HIS6_C253S_W16A | IL-12P35_C16S_C89S | 55.4 |
| IL-12P40-HIS6_C253S_L197A | IL-12P35_C16S_C89S | 54.9 |
| IL-12P40-HIS6_C253S_D94K | IL-12P35_C16S_C89S | 53.6 |
| IL-12P40-HIS6_C253S_K105E | IL-12P35_C16S_C89S | 54.1 |
| IL-12P40-HIS6_C253S_K196E | IL-12P35_C16S_C89S | 53.7 |
| IL-12P40-HIS6_C253S_K259Y | IL-12P35_C16S_C89S | 55.1 |
| IL-12P40-HIS6_C253S_K265Y | IL-12P35_C16S_C89S | 55.2 |
| P40HisC253S_W16AD94K | IL-12P35_C16S_C89S | 54.5 |
| P40HisC253S_W16AK196E | IL-12P35_C16S_C89S | 53.8 |
| P40HisC253S_D94KK196E | IL-12P35_C16S_C89S | 52.9 |
| P40HisC253S_W16AD94KK196E | IL-12P35_C16S_C89S | 53.4 |
| P40HisC253S_ | IL-12P35_C16S_C89S | 53.2 |
| W16AD94KK196EL197A | | |
| P40HisC253S_ | IL-12P35_C16S_C89S | 53.5 |
| W16AD94KK196EK105E | | |
| P40HisC253S_ | IL-12P35_C16S_C89S | 53.5 |
| W16AD94KK196EK259Y | | |
| P40HisC253S_ | IL-12P35_C16S_C89S | 53.7 |
| W16AD94KK196EK265Y | | |
| P40HisC253S_K105EK259Y | IL-12P35_C16S_C89S | 54.2 |
| P40HisC253S_K105EK265Y | IL-12P35_C16S_C89S | 54.1 |
| P40HisC253S_K259YK265Y | IL-12P35_C16S_C89S | 55.1 |
| P40HisC253S_ | IL-12P35_C16S_C89S | 54.1 |
| K105EK259YK265Y | | |

### Example 3

### Label-Free Kinetic Analysis with SPR of IL-12 Variants

This example describes methods to identify IL-12p70-6HIS variants that maintain binding to IL-12RB1 and IL-12RB2 extra cellular domains ECDs. Additionally, the impact of IL-12p70-6HIS pegylation on IL-12RB1 and IL-12RB2 ECD binding were also assessed. For each variant, the p40 subunit had the sequence of SEQ ID NO: 2 with the specified amino acid substitutions and a C-terminal 6His-tag, and the p35 subunit had the sequence of SEQ ID NO: 3 with the specified amino acid substitutions.

Anti-human antibody (Cytiva-AHC kit) were immobilized onto a CM4 chip (Cytiva) using amine coupling chemistry (from Amine Coupling Kit, Cytiva). The immobilization steps were carried out at a flow rate of 25 μL/minute in 1×HBS-EP+ buffer (Cytiva). The sensor surfaces were activated for 7 min with a mixture of NHS (0.05 M) and EDC (0.2 M). The AHC antibodies were injected over all flow cells used in the study at a concentration of 25 μg/mL in 10 mM sodium acetate, pH 5, for seven minutes. Ethanolamine (1 M, pH 8.5) was injected for seven minutes to block any remaining activated groups. An average of 4,500 response units (RU) of capture antibody was immobilized on each flow cell used in the study.

Kinetic binding experiments were performed at 25° C. using 1×HBS-EP+ buffer. IL-12RB1 hFc fusion or IL-12RB2 hFc fusions (R&D Systems) were injected over the AHC surface at concentrations of 25 μg/mL for 15 seconds at a flow rate of 10 μL/minute on flow cells 2 and 3 respectively, followed by a stabilization period for 30 seconds at the same flow rate. Kinetic characterization of conjugated or unconjugated IL-12p70-6HIS or variants was carried out in a range of concentrations from 0.1 to 30 nM depending on the expected kinetics and one injection of 0 nM analyte. After capturing ligand (IL-12RB1 hFc, IL-12RB2 hFc) on the AHC surface, the analyte (IL-12p70-6HIS variant) contact time was 180 seconds, followed by a 180 second dissociation time at a flow rate of 30 μL/min. Between each ligand capture and analyte binding cycle, regeneration was carried out using one injection of 3M MgCl2 (Cytiva. AHC Kit) for 60 seconds at 50 μL/minute and a 30 second stabilization period, followed by an injection of 3M MgCl2 for 30 seconds at 50 μL/minute and a 300 second stabilization period that ends the cycle. The data were fit with the Biacore T200 Evaluation software Kinetic Screen using a global fit 1:1 binding model with double reference subtraction and RI=Ymax/5. Tables 6-9 provide the ka, kd, and KD results for certain IL-12p70 variants.

TABLE 6
IL-12p70 variants with steering mutations in IL-12p40:
Binding to IL12-RB1

| p40 Description | p35 Description | ka (1/Ms) | kd (1/s) | KD (nM) |
|---|---|---|---|---|
| IL-12P40-HIS6_C253S | IL-12P35_C16S_C89S | 4.18E+07 | 1.83E−03 | 0.04 |
| IL-12P40-HIS6_C253S | IL-12P35_C16S_C89S | 4.02E+07 | 1.65E−03 | 0.04 |
| IL-12P40-HIS6_C253S_W16A | IL-12P35_C16S_C89S | 4.89E+07 | 4.90E−02 | NC |
| IL-12P40-HIS6_C253S_Y17A | IL-12P35_C16S_C89S | 4.92E+07 | 3.47E−03 | 0.07 |
| IL-12P40-HIS6_C253S_P21A | IL-12P35_C16S_C89S | 4.30E+07 | 2.40E−03 | 0.06 |
| IL-12P40-HIS6_C253S_D42A | IL-12P35_C16S_C89S | 2.42E+07 | 1.80E−03 | 0.07 |
| IL-12P40-HIS6_C253S_Q43A | IL-12P35_C16S_C89S | 4.19E+07 | 1.41E−03 | 0.03 |
| IL-12P40-HIS6_C253S_S44A | IL-12P35_C16S_C89S | 3.89E+07 | 1.93E−03 | 0.05 |
| IL-12P40-HIS6_C253S_S45A | IL-12P35_C16S_C89S | 4.33E+07 | 1.70E−03 | 0.04 |
| IL-12P40-HIS6_C253S_E46A | IL-12P35_C16S_C89S | 3.61E+07 | 2.20E−03 | 0.06 |
| IL-12P40-HIS6_C253S_K59A | IL-12P35_C16S_C89S | 4.29E+07 | 3.01E−03 | 0.07 |
| IL-12P40-HIS6_C253S_E87A | IL-12P35_C16S_C89S | 4.06E+07 | 1.75E−03 | 0.04 |
| IL-12P40-HIS6_C253S_D88A | IL-12P35_C16S_C89S | 3.43E+07 | 1.05E−03 | 0.03 |
| IL-12P40-HIS6_C253S_190A | IL-12P35_C16S_C89S | 3.24E+07 | 2.04E−03 | 0.06 |
| IL-12P40-HIS6_C253S_W91A | IL-12P35_C16S_C89S | 5.15E+07 | 3.42E−03 | 0.07 |
| IL-12P40-HIS6_C253S_D94A | IL-12P35_C16S_C89S | 9.41E+06 | 6.48E−03 | 0.69 |
| IL-12P40-HIS6_C253S_K103A | IL-12P35_C16S_C89S | 5.09E+07 | 2.23E−03 | 0.04 |
| IL-12P40-HIS6_C253S_N104A | IL-12P35_C16S_C89S | 3.69E+07 | 1.83E−03 | 0.05 |
| IL-12P40-HIS6_C253S_K105A | IL-12P35_C16S_C89S | 3.55E+07 | 2.27E−03 | 0.06 |
| IL-12P40-HIS6_C253S_D162A | IL-12P35_C16S_C89S | 3.38E+07 | 1.80E−03 | 0.05 |
| IL-12P40-HIS6_C253S_H195A | IL-12P35_C16S_C89S | 3.08E+07 | 4.09E−03 | 0.13 |
| IL-12P40-HIS6_C253S_K196A | IL-12P35_C16S_C89S | 1.81E+08 | 6.41E−03 | 0.04 |
| IL-12P40-HIS6_C253S_L197A | IL-12P35_C16S_C89S | 7.93E+07 | 4.03E−03 | 0.05 |
| IL-12P40-HIS6_C253S_N201A | IL-12P35_C16S_C89S | 7.01E+07 | 2.87E−03 | 0.04 |
| IL-12P40-HIS6_C253S_T203A | IL-12P35_C16S_C89S | 6.79E+07 | 2.53E−03 | 0.04 |
| IL-12P40-HIS6_C253S_D42K | IL-12P35_C16S_C89S | 6.46E+07 | 2.01E−03 | 0.03 |
| IL-12P40-HIS6_C253S_E46K | IL-12P35_C16S_C89S | 7.72E+07 | 3.29E−03 | 0.04 |
| IL-12P40-HIS6_C253S_K59E | IL-12P35_C16S_C89S | 7.62E+07 | 5.09E−03 | 0.07 |
| IL-12P40-HIS6_C253S_E87K | IL-12P35_C16S_C89S | 2.12E+07 | 2.32E−03 | 0.11 |
| IL-12P40-HIS6_C253S_D88K | IL-12P35_C16S_C89S | 3.06E+07 | 5.58E−04 | 0.02 |
| IL-12P40-HIS6_C253S_D94K | IL-12P35_C16S_C89S | 3.79E+07 | 2.83E−02 | 0.75 |
| IL-12P40-HIS6_C253S_K103E | IL-12P35_C16S_C89S | 3.50E+07 | 3.66E−03 | 0.11 |
| IL-12P40-HIS6_C253S_K105E | IL-12P35_C16S_C89S | 4.49E+07 | 6.00E−03 | 0.13 |
| IL-12P40-HIS6_C253S_D162K | IL-12P35_C16S_C89S | 4.07E+07 | 2.92E−03 | 0.07 |
| IL-12P40-HIS6_C253S_H195E | IL-12P35_C16S_C89S | 7.12E+07 | 3.93E−03 | 0.06 |
| IL-12P40-HIS6_C253S_K196E | IL-12P35_C16S_C89S | 6.91E+07 | 1.01E−02 | 0.15 |
| IL-12P40-HIS6_C253S_K259Y | IL-12P35_C16S_C89S | 7.59E+07 | 5.73E−03 | 0.08 |
| IL-12P40-HIS6_C253S_K265Y | IL-12P35_C16S_C89S | 9.93E+07 | 1.04E−02 | 0.11 |
| IL-12P40-HIS6_C253S_1278Y | IL-12P35_C16S_C89S | 5.41E+07 | 2.05E−03 | 0.04 |
| IL-12P40-HIS6_C253S_E87K, | IL-12P35_C16S_C89S | 6.74E+07 | 1.58E−02 | 0.24 |
| K196E | | | | |
| IL-12P40-HIS6_C253S_E87K, | IL-12P35_C16S_C89S | 4.12E+07 | 7.05E−02 | NC |
| K196E,K198E | | | | |

TABLE 7
IL-12p70 variants with steering mutations in IL-12p40:
Binding to IL12-RB2

| p40 Description | p35 Description | ka (1/Ms) | kd (1/s) | KD (nM) |
|---|---|---|---|---|
| IL-12P40-HIS6_C253S | IL-12P35_C16S_C89S | 8.95E+06 | 3.08E−03 | 0.75 |
| IL-12P40-HIS6_C253S | IL-12P35_C16S_C89S | 3.07E+06 | 2.30E−03 | 0.34 |
| IL-12P40-HIS6_C253S_W16A | IL-12P35_C16S_C89S | 6.45E+06 | 3.99E−03 | 0.62 |
| IL-12P40-HIS6_C253S_Y17A | IL-12P35_C16S_C89S | NC | NC | NC |
| IL-12P40-HIS6_C253S_P21A | IL-12P35_C16S_C89S | NC | NC | NC |
| IL-12P40-HIS6_C253S_D42A | IL-12P35_C16S_C89S | NC | NC | NC |
| IL-12P40-HIS6_C253S_Q43A | IL-12P35_C16S_C89S | NC | NC | NC |
| IL-12P40-HIS6_C253S_S44A | IL-12P35_C16S_C89S | NC | NC | NC |
| IL-12P40-HIS6_C253S_S45A | IL-12P35_C16S_C89S | NC | NC | NC |
| IL-12P40-HIS6_C253S_E46A | IL-12P35_C16S_C89S | NC | NC | NC |
| IL-12P40-HIS6_C253S_K59A | IL-12P35_C16S_C89S | 2.25E+06 | 4.81E−03 | 2.1 |
| IL-12P40-HIS6_C253S_E87A | IL-12P35_C16S_C89S | NC | NC | NC |
| IL-12P40-HIS6_C253S_D88A | IL-12P35_C16S_C89S | 9.45E+06 | 3.61E−03 | 0.38 |
| IL-12P40-HIS6_C253S_190A | IL-12P35_C16S_C89S | 1.00E+07 | 3.48E−03 | 0.35 |
| IL-12P40-HIS6_C253S_W91A | IL-12P35_C16S_C89S | 7.13E+06 | 3.80E−03 | 0.53 |
| IL-12P40-HIS6_C253S_D94A | IL-12P35_C16S_C89S | 1.12E+07 | 4.77E−03 | 0.43 |
| IL-12P40-HIS6_C253S_K103A | IL-12P35_C16S_C89S | 1.07E+07 | 3.56E−03 | 0.33 |
| IL-12P40-HIS6_C253S_N104A | IL-12P35_C16S_C89S | 9.60E+06 | 3.77E−03 | 0.39 |
| IL-12P40-HIS6_C253S_K105A | IL-12P35_C16S_C89S | 1.19E+07 | 3.99E−03 | 0.34 |
| IL-12P40-HIS6_C253S_D162A | IL-12P35_C16S_C89S | NC | NC | NC |
| IL-12P40-HIS6_C253S_H195A | IL-12P35_C16S_C89S | 9.17E+06 | 4.91E−03 | 0.54 |
| IL-12P40-HIS6_C253S_K196A | IL-12P35_C16S_C89S | 7.52E+06 | 4.38E−03 | 0.58 |
| IL-12P40-HIS6_C253S_L197A | IL-12P35_C16S_C89S | 7.05E+06 | 3.89E−03 | 0.55 |
| IL-12P40-HIS6_C253S_N201A | IL-12P35_C16S_C89S | 8.30E+06 | 4.14E−03 | 0.50 |
| IL-12P40-HIS6_C253S_T203A | IL-12P35_C16S_C89S | 7.97E+06 | 3.97E−03 | 0.50 |
| IL-12P40-HIS6_C253S_D42K | IL-12P35_C16S_C89S | 8.65E+06 | 3.87E−03 | 0.45 |
| IL-12P40-HIS6_C253S_E46K | IL-12P35_C16S_C89S | 7.58E+06 | 3.94E−03 | 0.52 |
| IL-12P40-HIS6_C253S_K59E | IL-12P35_C16S_C89S | 8.92E+06 | 4.35E−03 | 0.49 |
| IL-12P40-HIS6_C253S_E87K | IL-12P35_C16S_C89S | NC | NC | NC |
| IL-12P40-HIS6_C253S_D88K | IL-12P35_C16S_C89S | 1.12E+07 | 3.42E−03 | 0.31 |
| IL-12P40-HIS6_C253S_D94K | IL-12P35_C16S_C89S | 3.34E+06 | 3.95E−03 | 1.2 |
| IL-12P40-HIS6_C253S_K103E | IL-12P35_C16S_C89S | 1.05E+07 | 3.57E−03 | 0.34 |
| IL-12P40-HIS6_C253S_K105E | IL-12P35_C16S_C89S | 1.10E+07 | 6.17E−03 | 0.56 |
| IL-12P40-HIS6_C253S_D162K | IL-12P35_C16S_C89S | NC | NC | NC |
| IL-12P40-HIS6_C253S_H195E | IL-12P35_C16S_C89S | NC | NC | NC |
| IL-12P40-HIS6_C253S_K196E | IL-12P35_C16S_C89S | 1.18E+07 | 4.98E−03 | 0.42 |
| IL-12P40-HIS6_C253S_K259Y | IL-12P35_C16S_C89S | 5.67E+06 | 6.37E−03 | 1.1 |
| IL-12P40-HIS6_C253S_K265Y | IL-12P35_C16S_C89S | 4.94E+06 | 7.82E−03 | 1.6 |
| IL-12P40-HIS6_C253S_1278Y | IL-12P35_C16S_C89S | NC | NC | NC |
| IL-12P40-HIS6_C253S_E87K, | IL-12P35_C16S_C89S | 8.98E+06 | 4.39E−03 | 0.49 |
| K196E | | | | |
| IL-12P40-HIS6_C253S_E87K, | IL-12P35_C16S_C89S | 8.09E+06 | 4.97E−03 | 0.61 |
| K196E, K198E | | | | |

TABLE 8
IL-12p70 variants with multiple steering mutations in IL-12p40:
Binding to IL-12RB1

| p40 Description | p35 Description | ka (1/Ms) | kd (1/s) | KD (nM) |
|---|---|---|---|---|
| IL-12P40-HIS6_C253S | IL-12P35_C16S_C89S | 9.05E+06 | 1.02E−03 | 0.11 |
| IL-12P40-HIS6_C253S | IL-12P35_C16S_C89S | 1.53E+07 | 4.59E−03 | 0.30 |
| IL-12P40-HIS6_C253S_W16A | IL-12P35_C16S_C89S | NC | NC | NC |
| IL-12P40-HIS6_C253S_L197A | IL-12P35_C16S_C89S | 1.86E+07 | 5.73E−03 | 0.31 |
| IL-12P40-HIS6_C253S_D94K | IL-12P35_C16S_C89S | 6.97E+07 | 3.08E−02 | 0.44 |
| IL-12P40-HIS6_C253S_K105E | IL-12P35_C16S_C89S | 1.34E+07 | 6.66E−03 | 0.50 |
| IL-12P40-HIS6_C253S_K196E | IL-12P35_C16S_C89S | 1.49E+07 | 1.04E−02 | 0.70 |
| IL-12P40-HIS6_C253S_K259Y | IL-12P35_C16S_C89S | 9.23E+06 | 8.05E−03 | 0.87 |
| IL-12P40-HIS6_C253S_K265Y | IL-12P35_C16S_C89S | 1.34E+07 | 6.80E−03 | 0.51 |
| P40His C253S_W16AD94K | IL-12P35_C16S_C89S | NC | NC | NC |
| P40HisC253S_W16AK196E | IL-12P35_C16S_C89S | NC | NC | NC |
| P40HisC253S_D94KK196E | IL-12P35_C16S_C89S | NC | NC | NC |
| P40HisC253S_W16AD94KK196E | IL-12P35_C16S_C89S | NC | NC | NC |
| P40HisC253S_W16AD94KK196EL197A | IL-12P35_C16S_C89S | NC | NC | NC |
| P40HisC253S_W16AD94KK196EK105E | IL-12P35_C16S_C89S | NC | NC | NC |
| P40HisC253S_W16AD94KK196EK259Y | IL-12P35_C16S_C89S | NC | NC | NC |
| P40HisC253S_W16AD94KK196EK265Y | IL-12P35_C16S_C89S | NC | NC | NC |
| P40HisC253S_K105EK259Y | IL-12P35_C16S_C89S | NC | NC | NC |
| P40HisC253S_K105EK265Y | IL-12P35_C16S_C89S | 9.41E+06 | 1.07E−02 | 1.1 |
| P40HisC253S_K259YK265Y | IL-12P35_C16S_C89S | 9.86E+06 | 1.41E−02 | 1.4 |
| P40HisC253S_K105EK259YK265Y | IL-12P35_C16S_C89S | 7.66E+06 | 1.40E−02 | 1.8 |

TABLE 9
IL-12p70 variants with multiple steering mutations in IL-12p40:
Binding to IL-12RB2

| p40 Description | p35 Description | ka (1/Ms) | kd (1/s) | KD (nM) |
|---|---|---|---|---|
| IL-12P40-HIS6_C253S | IL-12P35_C16S_C89S | 1.84E+06 | 4.66E−03 | 2.5 |
| IL-12P40-HIS6_C253S | IL-12P35_C16S_C89S | 2.71E+06 | 6.37E−03 | 2.4 |
| IL-12P40-HIS6_C253S_W16A | IL-12P35_C16S_C89S | 8.78E+05 | 1.41E−02 | 16 |
| IL-12P40-HIS6_C253S_L197A | IL-12P35_C16S_C89S | 2.92E+06 | 6.81E−03 | 2.3 |
| IL-12P40-HIS6_C253S_D94K | IL-12P35_C16S_C89S | 3.20E+06 | 6.05E−03 | 1.9 |
| IL-12P40-HIS6_C253S_K105E | IL-12P35_C16S_C89S | 9.14E+06 | 9.61E−03 | 1.1 |
| IL-12P40-HIS6_C253S_K196E | IL-12P35_C16S_C89S | 2.77E+06 | 7.61E−03 | 2.7 |
| IL-12P40-HIS6_C253S_K259Y | IL-12P35_C16S_C89S | 2.30E+06 | 9.09E−03 | 4.0 |
| IL-12P40-HIS6_C253S_K265Y | IL-12P35_C16S_C89S | 1.90E+06 | 9.27E−03 | 4.9 |
| P40HisC253S_W16AD94K | IL-12P35_C16S_C89S | 2.62E+06 | 6.26E−03 | 2.4 |
| P40HisC253S_W16AK196E | IL-12P35_C16S_C89S | 2.83E+06 | 7.91E−03 | 2.8 |
| P40HisC253S_D94KK196E | IL-12P35_C16S_C89S | 2.89E+06 | 6.50E−03 | 2.3 |
| P40HisC253S_W16AD94KK196E | IL-12P35_C16S_C89S | 2.95E+06 | 6.74E−03 | 2.3 |
| P40HisC253S_W16AD94KK196EL197A | IL-12P35_C16S_C89S | 2.71E+06 | 8.05E−03 | 3.0 |
| P40HisC253S_W16AD94KK196EK105E | IL-12P35_C16S_C89S | NC | NC | NC |
| P40HisC253S_W16AD94KK196EK259Y | IL-12P35_C16S_C89S | 2.21E+06 | 4.07E−03 | 1.9 |
| P40HisC253S_W16AD94KK196EK265Y | IL-12P35_C16S_C89S | 2.02E+06 | 9.22E−03 | 4.6 |
| P40HisC253S_K105EK259Y | IL-12P35_C16S_C89S | NC | NC | NC |
| P40HisC253S_K105EK265Y | IL-12P35_C16S_C89S | 1.52E+07 | 2.44E−02 | 1.6 |
| P40HisC253S_K259YK265Y | IL-12P35_C16S_C89S | 1.54E+06 | 1.07E−02 | 7.0 |
| P40HisC253S_K105EK259YK265Y | IL-12P35_C16S_C89S | 4.32E+06 | 1.73E−02 | 4.0 |

### SEQUENCE LISTING

| SEQ | | |
|---|---|---|
| ID | | |
| NO: | Description | Amino Acid Sequence |
| 1 | IL-12 p40 (Uniprot | MCHQQLVISW FSLVFLASPL VAIWELKKDV |
| | P29460) | YVVELDWYPD APGEMVVLTC DTPEEDGITW |
| | | TLDQSSEVLG SGKTLTIQVK EFGDAGQYTC |
| | | HKGGEVLSHS LLLLHKKEDG IWSTDILKDQ |
| | | KEPKNKTFLR CEAKNYSGRE TCWWLTTIST |
| | | DLTFSVKSSR GSSDPQGVTC GAATLSAERV |
| | | RGDNKEYEYS VECQEDSACP AAEESLPIEV |
| | | MVDAVHKLKY ENYTSSFFIR DIIKPDPPKN |
| | | LQLKPLKNSR QVEVSWEYPD TWSTPHSYES |
| | | LTFCVQVQGK SKREKKDRVF TDKTSATVIC |
| | | RKNASISVRA QDRYYSSSWS EWASVPCS |
| 2 | IL-12 p40 (mature | MIWELKKDVY VVELDWYPDA PGEMVVLTCD |
| | form with N-terminal | TPEEDGITWT LDQSSEVLGS GKTLTIQVKE |
| | methionine) | FGDAGQYTCH KGGEVLSHSL LLLHKKEDGI |
| | | WSTDILKDQK EPKNKTFLRC EAKNYSGRET |
| | | CWWLTTISTD LTFSVKSSRG SSDPQGVTCG |
| | | AATLSAERVR GDNKEYEYSV ECQEDSACPA |
| | | AEESLPIEVM VDAVHKLKYE NYTSSFFIRD |
| | | IIKPDPPKNL QLKPLKNSRQ VEVSWEYPDT |
| | | WSTPHSYFSL TFCVQVQGKS KREKKDRVET |
| | | DKTSATVICR KNASISVRAQ DRYYSSSWSE |
| | | WASVPCS |
| 3 | IL-12 p35 (mature | MRNLPVATPD PGMFPCLHHS QNLLRAVSNM |
| | form with N-terminal | LQKARQTLEF YPCTSEEIDH EDITKDKTST |
| | methionine) | VEACLPLELT KNESCLNSRE TSFITNGSCL |
| | | ASRKTSEMMA LCLSSIYEDL KMYQVEFKTM |
| | | NAKLLMDPKR QIFLDQNMLA VIDELMQALN |
| | | FNSETVPQKS SLEEPDFYKT KIKLCILLHA |
| | | FRIRAVTIDR VMSYLNAS |
| 4 | IL-12 p35 | MWPPGSASQP PPSPAAATGL HPAARPVSLQ |
| | (NP 000873.2) | CRLSMCPARS LLLVATLVLL DHLSLARNLP |
| | | VATPDPGMFP CLHHSQNLLR AVSNMLQKAR |
| | | QTLEFYPCTS EEIDHEDITK DKTSTVEACL |
| | | PLELTKNESC LNSRETSFIT NGSCLASRKT |
| | | SEMMALCLSS IYEDLKMYQV EFKTMNAKLL |
| | | MDPKRQIFLD QNMLAVIDEL MCALNENSET |
| | | VPQKSSLEEP DFYKTKIKLC ILLHAFRIRA |
| | | VTIDRVMSYL NAS |
| 5 | IL-12 p35 | MWPPGSASQP PPSPAAATGL HPAARPVSLQ |
| | (NP 001341511.1) | CRLSMCPARS LLLVATLVLL DHLSLARNLP |
| | | VATPDPGMFP CLHHSQNLLR AVSNMLQKAR |
| | | QTLEFYPCTS EEIDHEDITK DKTSTVEACL |
| | | PLELTKNGSC LASRKTSEMM ALCLSSIYED |
| | | LKMYQVEFKT MNAKLLMDPK RCIFLDQNML |
| | | AVIDELMQAL NENSETVPQK SSLEEPDFYK |
| | | TKIKLCILLH AFRIRAVTID RVMSYLNAS |
| 6 | IL-12 p35 | MWPPGSASQP PPSPAAATGL HPAARPVSLQ |
| | (NP_001341512.1) | CRLSMCPARS LLLVATLVLL DHLSLARNLP |
| | | VATPDPGMFP CLHHSQNLLR AVSNMLQKNE |
| | | SCLNSRETSF ITNGSCLASR KTSEMMALCL |
| | | SSIYEDLKMY QVEFKTMNAK LLMDPKRQIF |
| | | LDQNMLAVID ELMQALNENS ETVPQKSSLE |
| | | EPDFYKTKIK LCILLHAFRI RAVTIDRVMS |
| | | YLNAS |
| 7 | IL-12 p40 | MIWELKKDVY VVELDWYPDA PGEMVVLTCD |
| | (mature form | TPEEDGITWT LDQSSEVLGS GKTLTIQVKE |
| | with N-terminal | FGDAGQYTCH KGGEVLSHSL LLLHKKEDGI |
| | methionine and C- | WSTDILKDQK EPKNKTFLRC EAKNYSGRFT |
| | terminal 6His) | CWWLTTISTD LTFSVKSSRG SSDPQGVTCG |
| | | AATLSAERVR GDNKEYEYSV ECQEDSACPA |
| | | AEESLPIEVM VDAVHKLKYE NYTSSFFIRD |
| | | IIKPDPPKNL QLKPLKNSRQ VEVSWEYPDT |
| | | WSTPHSYFSL TFCVQVQGKS KREKKDRVFT |
| | | DKTSATVICR KNASISVRAQ DRYYSSSWSE |
| | | WASVPCSGGG SHHHHHH |
| 8 | IL-12P40- | MIWELKKDVY VVELDWYPDA PGEMVVLTCD |
| | HIS6 C253S | TPEEDGITWT LDQSSEVLGS GKTLTIQVKE |
| | | FGDAGQYTCH KGGEVLSHSL LLLHKKEDGI |
| | | WSTDILKDQK EPKNKTFLRC EAKNYSGRFT |
| | | CWWLTTISTD LTFSVKSSRG SSDPQGVTCG |
| | | AATLSAERVR GDNKEYEYSV ECQEDSACPA |
| | | AEESLPIEVM VDAVHKLKYE NYTSSFFIRD |
| | | IIKPDPPKNL QLKPLKNSRQ VEVSWEYPDT |
| | | WSTPHSYFSL TESVQVQGKS KREKKDRVFT |
| | | DKTSATVICR KNASISVRAQ DRYYSSSWSE |
| | | WASVPCSGGG SHHHHHH |
| 9 | IL-12P35 | MRNLPVATPD PGMFPSLHHS QNLLRAVSNM |
| | C16S C89S | LQKARQTLEF YPCTSEEIDH EDITKDKTST |
| | | VEACLPLELT KNESCLNSRE TSFITNGSSL |
| | | ASRKTSEMMA LCLSSIYEDL KMYQVEFKTM |
| | | NAKLLMDPKR QIFLDQNMLA VIDELMQALN |
| | | FNSETVPQKS SLEEPDFYKT KIKLCILLHA |
| | | FRIRAVTIDR VMSYLNAS |

All publications, patents and patent applications cited in this specification are herein incorporated by reference as if each individual publication or patent application were specifically and individually indicated to be incorporated by reference.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of embodiments disclosed herein includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the present disclosure.

What is claimed:

1. A variant IL-12 p40 subunit polypeptide, wherein the variant IL-12 p40 subunit polypeptide comprises at least one amino acid substitution relative to SEQ ID NO: 1, wherein the at least one amino acid substitution is selected from the group consisting of: W16X, Y17A, P21A, D42A, D42K, Q43A, S44A, S45A, E46A, E46K, K59A, K59E, K85E, E87A, E87K, D88A, D88K, 190A, W91A, D94X, K103A, K103E, N104A, K105A, K105E, D162A, D162K, H195A, H195E, K196A, K196E, L197A, K198E, N201A, T203A, C253X, K259Y, K265Y, I278Y, and combinations thereof, wherein X indicates any amino acid other than the SEQ ID NO: 1 amino acid at the indicated position.

2. The variant IL-12 p40 subunit polypeptide of claim 1, wherein the at least one amino acid substitution is selected from the group consisting of: W16X, D94X, and K196E, wherein X indicates any amino acid other than the SEQ ID NO: 1 amino acid at the indicated position.

3. The variant IL-12 p40 subunit polypeptide of claim 1, wherein the at least one amino acid substitution is selected from the group consisting of: W16A, Y17A, P21A, D42A, D42K, Q43A, S44A, S45A, E46A, E46K, K59A, K59E, K85E, E87A, E87K, D88A, D88K, 190A, W91A, D94A, D94K, K103A, K103E, N104A, K105A, K105E, D162A, D162K, H195A, H195E, K196A, K196E, L197A, K198E, N201A, T203A, C253A, C253S, K259Y, K265Y, I278Y, and combinations thereof.

4. The variant IL-12 p40 subunit polypeptide of claim 1 or 3, wherein the at least one amino acid substitution is selected from the group consisting of: W16A/D94K; W16A/K196E; D94K/K196E; W16A/D94K/K196E; W16A/D94K/K196E/L197A; W16A/D94K/K196E/K105E; W16A/D94K/K196E/K259Y; W16A/D94K/K196E/K265Y; K105E/K259Y; K105E/K265Y; K259Y/K265Y; and K105E/K259Y/K265Y.

5. The variant IL-12 p40 subunit polypeptide of claim 1 or 3, wherein the at least one amino acid substitution is selected from the group consisting of: E87K/K196E, E87K/K198E, K196E/K198E and E87K/K196E/K198E.

6. The variant IL-12 p40 subunit polypeptide of claim 1 or 3, wherein the at least one amino acid substitution is selected from the group consisting of: W16A, K59E, K85E, E87K, D94A, D94K, K105E, K196A, K196E, L197A, K198E, K259Y, K265Y, E87K/K196E, E87K/K198E, K196E/K198E and E87K/K196E/K198E.

7. The variant IL-12 p40 subunit polypeptide of claim 1 or 3, wherein the variant IL-12 p40 subunit polypeptide comprises at least one amino acid substitution selected from the group consisting of: W16A, D94K, K105E, K196E, L197A, K259Y, and K265Y.

8. The variant IL-12 p40 subunit polypeptide of any one of claims 1-3, wherein the at least one amino acid substitution is selected from the group consisting of: W16A, D94K, and K196E.

9. The variant IL-12 p40 subunit polypeptide of claim 1 or 3, wherein the variant IL-12 p40 subunit polypeptide comprises less than five amino acid substitutions selected from the group consisting of: W16A, D94K, K105E, K196E, L197A, K259Y, and K265Y.

10. The variant IL-12 p40 subunit polypeptide of claim 6 or 7, wherein the at least one amino acid substitution is selected from the group consisting of: W16A, K59E, D94A, D94K, K196A, K196E, L197A, K198E, E87K, K196E, K198E, and combinations thereof.

11. The variant IL-12 p40 subunit polypeptide of claim 10, wherein the at least one amino acid substitution is selected from the group consisting of: E87K, K196E, K198E, and combinations thereof.

12. The variant IL-12 p40 subunit polypeptide of claim 6, wherein the at least one amino acid substitution is selected from the group consisting of: E87K/K196E, E87K/K198E, K196E/K198E and E87K/K196E/K198E.

13. The variant IL-12 p40 subunit polypeptide of any one of claims 1-10, wherein the variant IL-12 p40 subunit polypeptide comprises or further comprises a C253A substitution.

14. The variant IL-12 p40 subunit polypeptide of any one of claims 1-11, wherein the variant IL-12 p40 subunit polypeptide has at least 90% sequence identity to SEQ ID NO: 1 or SEQ ID NO: 2.

15. The variant IL-12 p40 subunit polypeptide of any one of claims 1-12, wherein the variant IL-12 p40 subunit polypeptide has reduced affinity for IL-12 receptor subunit beta-1 (IL-12Rβ1).

16. The variant IL-12 p40 subunit polypeptide of the any one of claims 1-13, wherein the at least one amino acid substitution is on an IL-12Rβ1 receptor contacting surface of the variant IL-12 p40 subunit polypeptide.

17. The variant IL-12 p40 subunit polypeptide of any one of claims 1-14, wherein the at least one amino acid substitution in the variant IL-12 p40 subunit polypeptide is located at an amino acid position that contacts IL-12Rβ1 through hydrogen bonds and/or ionic bonds.

18. The variant IL-12 p40 subunit polypeptide of any one of claims 1-15, wherein the at least one amino acid substitution in the variant IL-12 p40 subunit polypeptide is at a position that contacts IL-12Rβ1 through ionic bonds.

19. The variant IL-12 p40 subunit polypeptide of any one of claims 1-18, wherein the variant IL-12 p40 subunit polypeptide lacks an N-terminal methionine.

20. A nucleic acid encoding a variant IL-12 p40 subunit polypeptide of any one of claims 1-19.

21. A variant IL-12 cytokine comprising the variant IL-12 p40 subunit polypeptide of any one of claims 1-19 and an IL-12 p35 subunit polypeptide or a variant IL-12 p35 subunit polypeptide.

22. The variant IL-12 cytokine of claim 21, wherein the variant IL-12 p35 subunit polypeptide comprises at least one amino acid substitution of the variant IL-12 p35 subunit polypeptide is selected from the group consisting of C16X and C89X relative to SEQ ID NO: 3.

23. The variant IL-12 cytokine of claim 22, wherein the at least one amino acid substitution of the variant IL-12 p35 subunit polypeptide is selected from the group consisting of C16S and C89S.

24. The variant IL-12 cytokine of claim 20, wherein the variant IL-12 cytokine has reduced affinity for IL-12 receptor subunit beta-1 (IL-12Rβ1).

25. A composition comprising the variant IL-12 p40 subunit polypeptide of any one of claims 1-19, a variant IL-12 cytokine of any one of claims 21-24 and a pharmaceutically acceptable carrier or excipient.

26. The composition of claim 25, wherein the composition is used for treating cancer.

27. The composition of claim 26, wherein the cancer is selected from a solid tumor and an hematological malignancy.

28. A method for treating a disease, the method comprising administering a therapeutically effective amount of a variant IL-12 p40 subunit polypeptide of any one of claims 1-19, a variant IL-12 cytokine of any one of claims 21-24, or the composition of claim 25, to a subject in need thereof.

29. The method of claim 28, wherein the disease is cancer.

30. The method of claim 29, wherein the cancer is selected from a solid tumor and an hematological malignancy.

31. The method of claim 29, wherein the administration is systemic administration.

32. The method of claim 29, wherein the administration is by intratumoral injection.

33. The method of any one of claims 28 to 32, further comprising administering a second therapeutic agent.

34. The method of claim 33, wherein the second therapeutic agent is administered simultaneously with the variant IL-12 p40 subunit polypeptide, variant IL-12 cytokine or the composition.

35. The method of claim 33, wherein the second therapeutic agent and the variant IL-12 p40 subunit polypeptide, variant IL-12 cytokine or the composition are administered sequentially.

36. The method of claim 33, wherein the second therapeutic agent is selected from the group consisting of: a chemotherapeutic agent, one or more additional cytokines, a variant IL-12 p40 subunit, a variant IL-12 cytokine, an antiangiogenic agent, radiotherapy, adoptive therapy, and a tumor vaccine.

37. The method of claim 33, wherein the second therapeutic agent is an immune checkpoint inhibitor.

38. The method of claim 37, wherein the immune checkpoint inhibitor is selected from the group consisting of: an anti-PD-1 variant IL-12 p40 subunit, an anti-PD-LI variant IL-12 p40 subunit, an anti-TIM3 variant IL-12 p40 subunit, an anti-TIGIT variant IL-12 p40 subunit, an anti-LAG3 variant IL-12 p40 subunit, and an anti-CTLA-4 variant IL-12 p40 subunit.

39. A method of inducing T cell expansion in a patient in need thereof comprising administering a therapeutically effective amount of a variant IL-2 p40 subunit polypeptide of any one of claims 1-19, a variant IL-12 cytokine of any one of claims 21-24, or the composition of claim 25, to a subject in need thereof.
